(19) United States
(12) Patent Application Publication
(10) Pub. No.: US 2005/0260619 A1
(43) Pub. Date: Nov. 24, 2005

(54) DNA microarray for fingerprinting and characterization of microorganisms in microbial communities

(76) Inventors: Roland Brousseau, Montreal (CA); Jason Dubois, Montreal (CA); Tom Edge, Toronto (CA); Luke Masson, Dollard-des-Ormeaux (CA); Jack T. Trevors, Guelph (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/055,637

(22) Filed: Feb. 11, 2005

Related U.S. Application Data
(60) Provisional application No. 60/543,288, filed on Feb. 11, 2004.

(51) Int. Cl. C12Q 1/68; C12M 1/34

(52) U.S. Cl. 435/6; 435/287.2

(57) ABSTRACT

An array of nucleic acid probes is described for identifying and/or characterizing a microorganism. Methods are also described for detecting the presence of a microorganism in a sample, as well as determining its pathotype, using the array. Methods of assessing related infection and disease in a subject using the array are also described. Methods that characterize complex microbial communities using the array are also described.

### CROSS-REFERENCE TO RELATED APPLICATIONS

This application claim priority on prior application Ser. No. 60/453,288 filed Feb. 11, 2004, the entire content of which is hereby incorporated by reference.

### TECHNICAL FIELD

The present invention relates to a DNA array plate and uses thereof, and more particularly to an array for detecting or pathotyping a microorganism and uses thereof. The present invention also relates to the use of a DNA array plate to characterize complex microbial mixtures and microbial communities. The present invention also demonstrates the use of a DNA array plate to determine the presence of antibiotic resistance genes in complex microbial mixtures and communities.

### BACKGROUND OF THE INVENTION

Single species microbial products and complex microbial mixtures containing live microorganisms (consortia) are sold commercially and used by the general public and commercial biotechnology users. From a regulatory viewpoint, there are no easy methods to characterize these consortia in terms of the taxonomy and function of the microorganisms present. The presence or absence of microbial pathogens in these products is also difficult to assess. The presence of molecular and physiological mechanisms for antibiotic or metal resistance, which are of concern in terms of spreading these traits within the bacterial pathogens to which humans may be exposed, is also difficult to assess in these microbial products. The same problems are also found when analyzing microbial populations from biotechnology processes, water, air, soil or food samples. With reference to commercial microbial products characterization and quality control, the stability of microorganisms and their reproducibility between different batches or lots from the same supplier over a period of months to years is also difficult to assess with current methods.

Presently, basic microbiology methods relying on culturing methods followed by microscopic, morphological and biochemical tests are the principal approach to characterize the microorganism(s). Once the microorganisms are grown, methods such as fatty acid analysis by gas chromatography and mass spectrometry can be applied to identify the microbial species present. Additional microbiology methods can be used to study phenotypic traits of cultured microorganisms such antibiotic resistance, metal resistance, or catabolic properties. Newer molecular methods such as the Polymerase Chain Reaction (PCR) method can be used to detect certain specific genes of interest, for instance those genes coding for bacterial toxins, antibiotic resistance genes or catabolic properties (e.g. lipases, proteases, cellulases) that are suspected of being present in a given microbial sample under study. Electrophoresis methods like such as Denaturing Gradient Gel Electrophoresis (DGGE) can be used to provide genetic fingerprints and estimates of the microbial diversity present in a given consortium.

The methods are generally complex, time-consuming and applicable only to suspected pathogens or genes of specific concern in a given sample. They are also notoriously inadequate for characterizing microbial diversity since perhaps as many as 95-99% of microorganisms in complex microbial communities cannot be cultured with existing media and methods used in laboratories. Culture methods show inherent variability in their ability to grow bacteria that have been stored for weeks or months prior to use. Also, viable but nonculturable (VBNC) microorganisms cannot be grown and detected. Electrophoretic methods such as DGGE are labor-intensive and require skilled research personnel to achieve reproducible results. They also only provide a rough genetic fingerprint of microbial diversity in a consortia, and another overly labour-intensive DNA sequencing step is required to learn more about the taxonomic composition and potential presence of pathogens in complex consortia. While PCR methods can be applied to detecting specific microorganisms or genes of interest in consortia, independent PCR tests for many different microorganisms and genes can quickly become overly complicated and cost prohibitive.

It would be highly desirable to be provided with a DNA array capable of characterizing and even discriminating between multiple microorganism species in a sample, all in one assay.

### SUMMARY OF THE INVENTION

One aim of the present invention is to provide a DNA array capable of characterizing and even discriminating between multiple microorganism species in a sample, all in one assay.

Another aim of the present invention is to provide a method for characterizing numerous microorganism in a same assay.

According to one aspect of the invention, a DNA microarray is provided including immobilized probes capable of providing a genetic fingerprint of a single species or of a consortia of microorganisms of interest in diverse water, soil, food, environmental and clinical samples, and recognizing specifically and simultaneously the presence therein of a plurality of gene classes such as:
a) Taxonomically significant genes such as 16S genes, heat shock proteins, RNA polymerase, DNA gyrase. Through a judicious selection of probes, information can be obtained on the presence or absence of diverse and similar microorganisms of environmental or human health relevance in a parallel, simultaneous fashion.
b) Functionally significant genes such as lipases, cellulases or proteases. As an example, a drain-cleaning microbial consortium lacking the stable presence of known lipase genes may be suspected of poor or inconsistent efficacy.
c) Genes of clinical interest to humans, wild animals, pets, livestock, insects, plants, biocontrol agents, such as antibiotic resistance genes,
d) Genes coding for known virulence factors, growth factors and toxins, to protect against inadvertent or deliberate contamination of a microbial product or process by pathogenic agents.
e) Any other gene of interest such as genes coding for specific proteins or macromolecules, cell components, waste products and antimicrobial agents.

According to yet another aspect of the invention, a method for providing a genetic fingerprint of a single species or of consortia of microorganisms in diverse water, soil, food, environmental and clinical samples, and recognizing specifically and simultaneously the presence therein of a plurality of gene classes, is provided comprising, a) extracting the total DNA from a microbial sample, b) labeling the sample with a detectable label and c) applying the labeled sample to a DNA microarray, wherein specific hybridization will occur with the relevant probes or oligonucleotides printed on the DNA microarray, d) reading the microarray with means appropriate to detect the detectable label (whether radioactive, non-radioactive, fluorescent, calorimetric, immunological, enzymatic, spectrophotometric or simply by visual detection with the unaided eye or through a microscope), to provide information simultaneously on all the types of probes mentioned above as to whether the sample contains or not sequences complementary to the probes printed on the DNA microarray.

According to a further aspect of the invention, there is also provided a microarray comprising thereon cpn60 probes and other useful probes such as 16S, antibiotic resistance, virulence genes, functional genes, for characterization of commercial microbial consortia. While the use of 16S is universal for species identification, its closely conserved nature leads to difficulties when closely related species are considered, such as Bacillus megaterium and Bacillus licheniformis (8% distance between the 16S gene sequences). Under those circumstances, the use of a more rapidly evolving gene such as cpn60 gives more differentiation.

According to yet a further aspect of the invention, the use of the microarrays according to the invention in the characterization of microbial communities in food microbiology, soil microbiology, water quality analysis, bio-terrorism detection, microbial air quality and similar applications, is also provided.

In accordance with the present invention, thereis also provided an array which comprises:
a) a substrate; and
b) a plurality of nucleic acid probes specifically and simultaneously recognizing the presence of a plurality of different genes, each of said probes being bound to said substrate at a discrete location; said plurality of probes comprising a first probe for detecting a first gene of a species of a microorganism and at least another probe for detecting at least one other gene of said species or of a different species of a microorganism.

Preferably, the array comprises at least two different probes specific for a single gene. The array may have a subarray containing said at least two probes at adjacent discrete locations on said substrate.

In one embodiment of the invention, the first probe as described above is specific for a virulence gene or a fragment thereof or a sequence substantially identical thereto, and the at least one other probe is specific for an antibiotic resistance gene. Alternatively, the first probe can be specific for a variant of a virulence gene or a fragment thereof or a sequence substantially identical thereto, and the at least one other probe is specific for an antibiotic resistance gene, the first probe allowing detection of different types and/or species of microorganism.

The microorganism can be a bacterium, and more particularly one of the family Enterobacteriaceae, such as E. coli.

In a further embodiment, the virulence gene encodes a polypeptide of a class of proteins selected from the group consisting of toxins, adhesion factors, secretory system proteins, capsule antigens, somatic antigens, flagellar antigens, invasins, autotransporter proteins, and aerobactin system proteins. In another embodiment of the invention, the different genes can be selected from the group consisting of Tem, Shv, oxa-1, oxa-7, pse-4, ctx-m, ant(3″)-Ia (aadA1), ant(2″)-Ia (aadB)b, aac(3)-IIa (aacC2), aac(3)-IV, aph(3′)-Ia (aphA1), aph(3′)-IIa (aphA2), tet(A), tet(B), tet(C), tet(D), tet(E), tet(Y), catI, catII, catIII, floR, dhfrI, dhfrV, dhfrVII, dhfrIX, dhfrXIII, dhfrXV, suII, suIII, integron classe 1 3′-CS, vat, vatC, vatD, vatE, vga, vgb, and vgbB.

Preferably, in one further embodiment of the invention, the plurality of nucleic acid probes are sequences selected from the group consisting of SEQ ID NO:1 to SEQ ID NO:64, or a fragment thereof, or a sequence having at least 50% identity, preferably at least 70% identity, more preferably having 80% identity and most preferably having 90% identity with said sequences.

The plurality of different genes can also be selected from the group consisting of 16S gene, genes encoding heat shock proteins, gene encoding RNA polymerase, gene encoding DNA gyrase, gene encoding a lipase, gene encoding a cellulose, gene encoding a protease, genes of clinical interest, gene encoding virulence factor, gene encoding growth factor, and gene encoding a toxin.

In a still further embodiment of the invention, the first probe is specific for a 16S gene or a fragment thereof or a sequence substantially identical thereto, and the at least one other probe is specific for cpn60 gene.

Also, in accordance with the present invention, there is provided a method of detecting the presence of a microorganism in a sample. The method comprises the steps of:
a) contacting an array as described above with a sample nucleic acid of said sample; and
b) detecting association of said sample nucleic acid to a probe on said array;
wherein association of said sample nucleic acid with said probe is indicative that said sample comprises a microorganism from which the nucleic acid sequence of said probe is derived.

The method may also comprise optionally a step of extraction of the sample nucleic acid from said sample prior to contacting said sample nucleic acid with said array.

The sample can be an environmental sample (such as from water, air or soil), a biological sample (such as blood, urine, amniotic fluid, feces, tissues, cells, cell cultures and biological secretions, excretions or discharge) or a food sample. Alternatively, the biological sample can be a tissue, body fluid, secretion or excretion from a subject.

In accordance with a further embodiment of the invention, there is also provided a method for determining a pathotype of a species of a microorganism in a sample, said method comprising the steps of:
a) contacting the array as defined previously with a sample nucleic acid of said sample; and
b) detecting association of said sample nucleic acid to a probe on said array;
wherein association of said sample nucleic acid with said probe is indicative that said sample having a pathotype from which the nucleic acid sequence of said probe is derived.

Still in accordance with the present invention, there is also provided a method for diagnosing an infection by a microorganism in a subject, said method comprising the steps of:
a) contacting the array as defined previously with a sample nucleic acid of said sample; and
b) detecting association of said sample nucleic acid to a probe on said array;
wherein association of said sample nucleic acid with said probe is indicative that said sample has been infected by a microorganism from which the nucleic acid sequence of said probe is derived.

According to the present invention, there is also provided a kit comprising the array as described above together with instructions for use thereof, such as uses for
(a) detecting the presence of a microorganism in a sample;
(b) determining the pathotype of a microorganism in a sample;
(c) diagnosing an infection by a microorganism in a subject; or
(d) diagnosing a condition related to infection by a microorganism, in a subject.

(e) characterizing a microbial complex sample or microbial community on a one-time basis
(f) following the evolution over time of a microbial complex sample or microbial community. This may include comparison between different batches of commercial products based on complex microbial samples, comparison between similar products from different suppliers and monitoring the bacterial composition of commercial products over storage time.

### INDUSTRIAL APPLICABILITY

The method proposed is generally applicable to any sample requiring microbiological analysis, such as:
i. single microbial species, clinical samples, commercial microbial consortia and communities of microorganisms from air, water and soil;
ii. food, food samples, food ingredients, livestock and pet food and the raw ingredients for making such foods;
iii. cosmetics, medications, pharmaceutical products and the raw ingredients to make such products;
iv. wastewater samples, potable water, raw water, surface water, groundwater, water treatment facilities, sewage samples;
v. bioreactor samples;
vi. human and veterinary clinical samples such as fecal or urine samples, animal tissue samples, rumen or stomach samples;
vii. plants, seeds, roots, plant surfaces, plant transplants, horticultural samples, nutrient recycling samples, plant rhizosphere, plant rhizoplane;
viii. environmental surfaces;
ix. samples from the manufacture or production of biological products, microorganisms, insects, protozoa;
x. goods produced in controlled atmosphere such as medical devices or electronics components; and
xi. any other samples where microorganisms can be detected and sampled.

An initial application of the invention resides in assisting biotechnology companies to meet notification requirements for consortia products under the Canadian Environmental Protection Act within Environment Canada. Commercial application may also be found within contract research or quality control laboratories. This invention could also be used for detection and/or identification of biological warfare agents camouflaged as commercial products. The invention can also be applied to any type of single microbial species or complex microbial consortium or mixture, within detection limits and given the design of suitable probes for each particular consortium. Therefore, companies that specialize in the detection and identification of microorganisms may also be interested. Also, companies involved and microbiological aspects of environmental, air quality and food monitoring, whether in consulting, R&D, quality control or research are expected to show interest worldwide. Basic research laboratories throughout the world will also be interested in the present invention.

For the purpose of the present invention the following terms are defined below.

The term “probe” is used herein interchangeably with amplicon and oligonucleotide of at least 18 or more nucleotides in length and preferably of at least 70 nucleotides in length.

The term “array” used herein is interchangeably used with the expression “array plate” or “DNA chip”.

The term “specific for” when used to set a probe in relation to a gene is intended to mean that said probe recognizes only the gene for which the probe is specific. Of course, a skilled person will appreciate that probes with silent substitutions, deletions or additions may as well be used in accordance with the present invention.

### BRIEF DESCRIPTION OF THE DRAWINGS

FIG. 1A illustrates a number of bacteria and antibiotic resistant bacteria present in commercial consortia, grown in LB media alone or in LB media containing Ampicillin, Chloramphenicol, Kanamycin, Streptomycin or Tetracycline at 25 or 50 μg/mL concentration;

FIG. 1B illustrates the direct detection of antibiotic resistance genes in genomic DNA extracted from a commercial consortium, the sequences of the probes detecting antibiotic resistance are found in Table 1;

FIG. 2 illustrates the detection results obtained on Biozyme 5000 commercial product, wherein solid yellow box represents an expected signal, the dashed red box represents samples known to cross react and dashed orange box represents a possible signal, the content being reported in Table 2;

FIGS. 3A to 3C illustrate the content (3A) printed on the microarray plates and the discriminating power of cpn60 probes between B38 B. megaterium (500 ng of DNA) (3B) and B16 B. licheniformis (500 ng of DNA) (3C);

FIGS. 4A and 4B illustrate the Key for the amplicon microarray used to illustrate the superior discriminating power of cpn60 genes between closely related species (4A) and the detection results (4B) of Amphibacillus xylanus using a combination of cpn60 and 16S probes;

FIG. 5 illustrates the detection results of Bacillus amyloliquefaciens using a combination of cpn60 and 16S probes as set out in Table 3, using the key illustrate in FIG. 4A;

FIG. 6 illustrates the detection results of Halobacillus halophilus using a combination of cpn60 and 16S probes as set out in Table 3, using the key illustrate in FIG. 4A; and

FIG. 7 illustrates the detection results of Virgibacillus pantothenticus using a combination of cpn60 and 16S probes as set out in Table 3, using the key illustrate in FIG. 4A.

### DETAILED DESCRIPTION OF THE PREFERRED EMBODIMENT

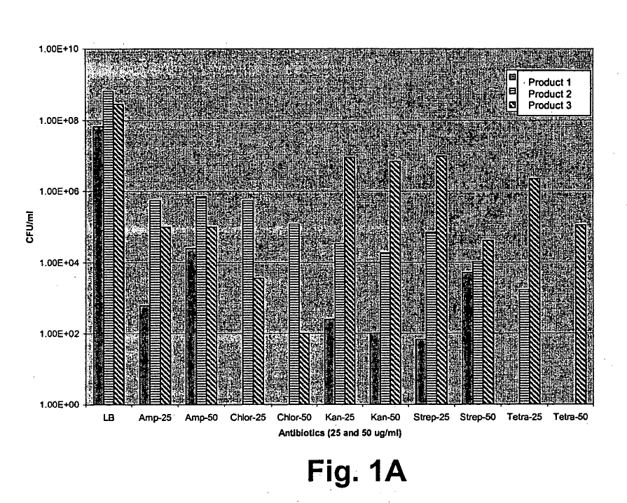

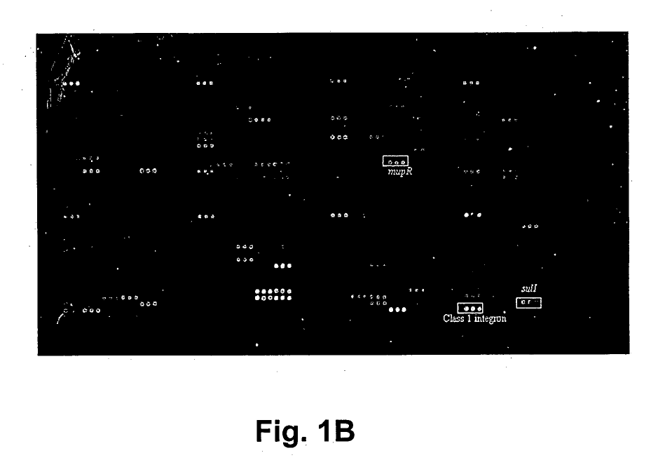

One of the concerns associated with the increasing occurrence of highly antibiotic resistant pathogenic bacteria in hospitals has been to find ways to slow the circulation of the resistance genes. In this context, increased use of microbial biotechnology products, particularly in consumer household environments, may be a concern if these products are found to contain medically significant antibiotic resistance genes. As shown in FIG. 1, commercially available microbial consortia in Canada have been found to contain high counts of antibiotic resistant bacteria.

To address these and other antibiotic resistance genes that may be present in commercial consortia, the inventors have developed antibiotic resistance gene probes for the consortium analysis microarray. The current design uses both oligonucleotides (18 to 70 bases) and amplicons as probes, to obtain the best trade-offs in sensitivity versus specicifity.

Presently, prokaryotic taxonomy is based, in part, upon sequence differences in the gene encoding 16S ribosomal RNA. This ordering makes sense for the most part and allows the discrimination of general taxonomic groups. However, within a narrow taxonomic group such as within a particular genus, 16S becomes less reliable as a taxonomic discriminator. Other genes, such as cpn60, a gene that encodes a 60 kDa chaperonin found in all bacteria, can also be used to delineate taxonomic lines due to its greater sequence diversity than 16S. A DNA microarray was printed with a combination of amplicon probes containing the sequences of 16S and cpn60 from a number of Bacillus and Bacillus-like species. The array was then hybridized with fluorescently-labelled amplicons of 16S and cpn60 amplified from different species that were represented on the microarray. The aim of the current work was to: 1) ascertain the validity of using such a dual taxonomic factor approach for discriminating between closely-related Bacillus species, and 2) determine whether the level of target discrimination required was achievable using DNA microarrays. The results confirm the complementarity that exists through the concomitant use of both taxonomic factors, and the parallel processing inherent in DNA microarrays, makes it a powerful tool to rapidly identify bacterial isolates at the species level.

The temperature at which a hybridization is carried out appears to be a major factor in achieving specificity. The 16S and cpn60 amplicons are of similar length (520-550 bp), but the 16S amplicons have a significantly higher GC content (57%) and melting temperature than the cpn60 amplicons (44%). This makes simultaneous hybridizations of the two amplicons on the same array less than optimal. However, by hybridizing at 55° C., a temperature between the optimum for each type of amplicon, signal discrimination for the cpn60 and 16S probes was obtained.

Due to the relatively small differences in sequence amongst the 16S probes printed on the array, some cross hybridization is expected. However, cross hybridizing signal should be proportional to the sequence similarity between the probe and target. The role of the 16S probes was to discriminate between different genera of bacteria, such as Halobacillus and Bacillus, while the cpn60, due to its greater variation, could discriminate at the species level.

The concept of a dual backbone microarray assay for the taxonomic discrimination of closely related bacteria was proven to work with amplicon hybridizations. Further work will examine whether this is valid with genomic DNA hybridizations.

The following tables 1 to 3 give a summary of the current status of probe development:

TABLE 1

70-mer oligonucleotide probes for commonly
encountered antibiotic resistance genes in bacteria

| | | | | | | | | |
|---|---|---|---|---|---|---|---|---|
| | | Length | | | | BLAST result, | | |
| | | of | | | G + C | (mm = | Accession | |
| Gene | Oligo probe (5′to 3′) | sequence | Position | Tm | content | mismatches) | number | oligo name |

Gram-
negative

| | | | | | | | | | |
|---|---|---|---|---|---|---|---|---|---|
| tem | AAA GTT CTG CTA TGT GGC GCG | 70 | 8674- | 80.4 | 57.1 | tem(X) | AF307748 | 70-tem8674 | |
| | GTA TTA TCC CGT GTT GAC GCC | | 8605 | | | | | | |
| | GGG CAA GAG CAA CTC GGT CGC | | | | | | | | |
| | CGC ATA C (SEQ ID NO:1) | | | | | | | | |
| shv | CTC AAG CGG CTG CGG GCT GGC | 70 | 86-17 | 83.7 | 64.3 | shv(X) | AF148850 | 70-shv86 | |
| | GTG TAC CGC CAG CGG CAG GGT | | | | | | | | |
| | GGC TAA CAG GGA GAT AAT ACA | | | | | | | | |
| | CAG GCG A (SEQ ID NO:2) | | | | | | | | |
| oxa-1 | AAA CAA CCT TCA GTT CCT TCA | 70 | 256-187 | 74.3 | 44.3 | oxa-1 | AJ238349 | 70- | |
| | AAT AAT GGA GAT GCG ACA GTA | | | | | | | oxa(1)256 | |
| | GAG ATA TCT GTT GAT GCA CTG | | | | | | | | |
| | GCG CTG C (SEQ ID NO:3) | | | | | | | | |
| oxa-7 | GTA GCG CAG GCT AAT TTA CTG | 70 | 295-226 | 75.2 | 45.7 | oxa-13, oxa-19, | X75562 | 70 | |
| | CTA CTT TTA CAA AGC ACG AAA | | | | | oxa-14, pse-2, | oxa(7)295 | | |
| | ACA CCA TTG ACG GCT TCG GCA | | | | | oxa-10, oxa-17, | | | |
| | GAG AAC T (SEQ ID NO:4) | | | | | oxa-16, oxa-7 | | | |
| pse-4 | CGC TGA TTG CCA TTG TAA TCC | 70 | 348-79 | 72.3 | 41.4 | pse-4, pse-5, | J05162 | 70- | |
| | CAA TAT TCT CCA TTT TGA GTA | | | | | carb-6, pse-1 | | pse(4)348 | |
| | TCA AGA ACG GAA ACA CCT ATA | | | | | | | | |
| | CGA GCA G (SEQ ID NO:5) | | | | | | | | |
| ctx-m | ATA CAG CGG CAC ACT TCC TAA | 70 | 143-74 | 80.3 | 55.7 | ctx-m-1, ctx-m-3, | X92506 | 70-ctx143 | |
| | CAA CAG CGT GAC GGT TGC CGT | | | | | ctx-m-28, ctx-m-, | | | |
| | CGC CAT CAG CGT GAA CTG ACG | | | | | 27, ctx-m-22, | | | |
| | CAG TGA (SEQ ID NO:6) | | | | | ctx-m-27, ctx- | | | |
| | | | | | | m-15 | | | |
| ant- | ATG ATG TCG TCG TGC ACA ACA | 70 | 290-221 | 79.2 | 55.7 | aadA1, aadA2 | X12870 | 70- | 70- |
| (3“)-la | ATG GTG ACT TCT ACA GCG CGG | | | | | | | aadA(1)290 | |
| (andA1) | AGA ATC TCG CTC TCT CCA GGG | | | | | | | | |
| | GAA GCC G (SEQ ID NO:7) | | | | | | | | |
| ant- | CCC GAG TGA GGT GCA TGC GAG | 70 | 1778- | 79.1 | 55.7 | aadB | M86913 | 70- | |
| (2“)-la | CCT GTA GGA CTC TAT GTG CTT | | 1709 | | | | | aadB1778 | |
| (aadB)^{b} | TGT AGG CCA GTC CAC TGG TGG | | | | | | | | |
| | TAC TTC A (SEQ ID NO:8) | | | | | | | | |
| aac(3)- | CAC CGG TTT GGA CTC CGA GTT | 70 | 200-131 | 77.7 | 52.3 | aacC2 | S68058 | 70- | |
| IIa | TTC GAA TTG CCT CCG TTA TTG | | | | | | | aacC(2)200 | |
| (aacC2) | CCT TCC GCG TAT GCA TCG CGA | | | | | | | | |
| | TAT CTC C (SEQ ID NO:9) | | | | | | | | |
| aac(3)- | TCG ATC AGT CCA AGT GGC CCA | 70 | 380-311 | 82.7 | 62.9 | aac(3)-IV | X01385 | 70- | |
| IV | TCT TCG AGG GGC CGG ACG CTA | | | | | | | aac3(IV)380 | |
| | CGG AAG GAG CTG TGG ACC AGC | | | | | | | | |
| | AGC ACA C (SEQ ID NO:10) | | | | | | | | |
| aph(3′)- | GGC GCA TCG GGC TTC CCA TAC | 70 | 1310- | 79.1 | 54.3 | aphA1, aphA7, | V00359 | 70 | |
| Ia | AAT CGA TAG ATT GTC CCT GAT | | 1241 | | | strA, | | aphA(1)1310 | |
| (aphA1) | TGC CCG ACA TTA TCG CGA GCC | | | | | Tn903 | | | |
| | CAT T (SEQ ID NO:11) | | | | | | | | |
| aph(3′)- | AGT CAT AGC CGA ATA GCC TCT | 70 | 220-151 | 78.9 | 52.9 | Tn5, aphA2, | V00618 | 70- | |
| Ila | CCA CCC AAG CGG CCG GAG AAC | | | | | aph(3′) | | aphA(2)220 | |
| (aphA2) | CTG CGT GCA ATC CAT CTT GTT | | | | | | | | |
| | CAA TCA T (SEQ ID NO:12) | | | | | | | | |
| tet(A) | GAT GCC GAC AGC GTC GAG CGC | 70 | 1390- | 79.5 | 57.1 | tetA | X00006 | 70-tetA1390 | |
| | GAC AGT GCT CAG AAT TAC GAT | | 1321 | | | | | | |
| | CAG GGG TAT GTT GGG TTT CAC | | | | | | | | |
| | GTC TGG C (SEQ ID NO:13) | | | | | | | | |
| tet(B) | CAA AGT GGT TAG CGA TAT CTT | 70 | 190-121 | 71.8 | 40 | tetB,Tn10 | V00611 | 70-tetB190 | |
| | CCG AAG CAA TAA ATT CAC GTA | | | | | | | | |
| | ATA ACG TTG GCA AGA CTG GCA | | | | | | | | |
| | TGA TAA G (SEQ ID NO:14) | | | | | | | | |
| tet(C) | GAC TGG CGA TGC TGT CGG AAT | 70 | 130-61 | 80.8 | 58.6 | pBR322, RP1, | J01749 | 70-tetC130 | |
| | GGA CGA TAT CCC GCA AGA GGC | | | | | tetC... | | | |
| | CCG GCA GTA CCG GCA TAA CCA | | | | | | | | |
| | AGC CTA T (SEQ ID NO:15) | | | | | | | | |
| tet(D) | CAA ACG CGG CAC CCG CCA GGG | 70 | 1770- | 83.5 | 64.3 | tetA | X65876 | 70-tetD1770 | |
| | ATA ACA GCA GCA CCG GTC TGC | | 1701 | | | | | | |
| | GCC CCA GCT TAT CTG ACC ATC | | | | | | | | |
| | TGC CCA G (SEQ ID NO:16) | | | | | | | | |
| tet(E) | GTT GAG GCT GCA ACA GCT CCA | 70 | 370-301 | 78 | 51.4 | tetE | L06940 | 70-tetE370 | |
| | GTC GCA CCG GTA ATA CCA GCA | | | | | | | | |
| | ATT AAG CGT CCC AAA TAC AAC | | | | | | | | |
| | ACC CAC A (SEQ ID NO:17) | | | | | | | | |
| tet(Y) | TTA ATA AAG CCG GAA CCA CCG | 70 | 1770- | 76.5 | 47.1. | tetY | AF070999 | 70-tetY1770 | |
| | GCA TGA TTA ATC CCA AAC CAA | | 1701 | | | | | | |
| | TCG CAT CAA GCG CGA CAA | | | | | | | | |
| | CAA TGA GTG C (SEQ ID NO:18) | | | | | | | | |
| catI | TTT ACG GTC TTT AAA AAG GCC | 70 | 550-481 | 73.1 | 41.1 | cam, Tn9, R100, | M62822 | 70-cat550 | |
| | GTA ATA TCC AGC TGA ACG GTC | | | | | cat,... | | | |
| | TGG TTA TAG GTA CAT TGA GCA | | | | | | | | |
| | ACT GAC T (SEQ ID NO:19) | | | | | | | | |
| catII | AGC GGT AAT ATC GAG TTT GGT | 70 | 300-231 | 75.6 | 45.7 | catII | X53796 | 70-cat(2) | |
| | GGT CAG GCT GAA TCC GCA TTT | | | | | | 300 | | |
| | AAT CTG CTG ACG ATA AAG GGC | | | | | | | | |
| | AAA GTG T (SEQ ID NO:20) | | | | | | | | |
| catIII | TTT GCT TGT TAA GCT AAA ACC | 70 | 370-301 | 74.4 | 41.4 | catIII | X07848 | 70-cat(3) | |
| | ACA TGG TAA ACG ATG CCG ATA | | | | | | 370 | | |
| | AAA CTC AAA ATG CTC ACG GCG | | | | | | | | |
| | AAC CCA A (SEQ ID NO:21) | | | | | | | | |
| floR | GAC AAA GGC CGG TGC AGT TGA | 70 | 384-315 | 82.3 | 60 | floR, pp-flo | AF252855 | 70-floR384 | |
| | AGA CCA AGC TGC TCC CAG AGA | | | | | | | | |
| | CGC AAT GAC GAA AGC CGT TGC | | | | | | | | |
| | GCC CGC A (SEQ ID NO:22) | | | | | | | | |
| dhfr1 | GGT TAA AGC ATC TTT AAT TGA | 70 | 490-421 | 69.2 | 32.9 | dhfrl, Tn9 | X00926 | 70- | |
| | TGG AAA GAT CAA TAC GTT CTC | | | | | | | dhfr(1)490 | |
| | ATT GTC AGA TGT AAA ACT TGA | | | | | | | | |
| | ACG TGT T (SEQ ID NO:23) | | | | | | | | |
| dhfrV | GTA CAT GGC CTC TTC GAT CGA | 70 | 1560- | 76.6 | 51.4 | dhfrV,dhfrlb, | X12868 | 70- | |
| | CGG GAA TAC TAT TAC GTT GTC | | 1491 | | | dhfrXVI | | dhfr(5)1560 | |
| | ATT ATG GGC CGT CCA GGC TGA | | | | | | | | |
| | GCG AGT A (SEQ ID NO:24) | | | | | | | | |
| dhfrVII | GAA CAC CCA TAG AGT CAA ATG | 70 | 753-684 | 64.2 | 72.4 | dhfrVII, dhfrXVII | X58425 | 70- | |
| | TTT TCC TTC CAA CAA GGA GCC | | | | | | | dhfr(7)753 | |
| | ACT GAT TAT ATG TGA GCG CTT | | | | | | | | |
| | TAA AGA G (SEQ ID NO:25) | | | | | | | | |
| dhfrIX | AGC TTT GAA GTG TTT TAA ATC | 70 | 830-761 | 72.5 | 40 | dhfrlX | X57730 | 70- | |
| | TTG TGG TTC ATG CCA CGG AAT | | | | | | | dhfr(9)830 | |
| | CTG ATT TTC AAA TCC GAT ACC | | | | | | | | |
| | TCC TGT C (SEQ ID NO:26) | | | | | | | | |
| dhfrXIII | TGG CGC GAG AGC ACC ACT GTG | 70 | 929-860 | 82.1 | 58.6 | dhfrXIII | Z50802 | 70- | |
| | TGG CGG TTT GGT AAG GGC TTG | | | | | | | dhfr(1 3)929 | |
| | CCT ATG GAC TCA AAT GTC TTG | | | | | | | | |
| | CGG CCC A (SEQ ID NO:27) | | | | | | | | |
| dhfrXV | CTT CAG ATG ATT TAG CGC TTC | 70 | 620-551 | 71.2 | 38.6 | dhfrXV | Z83311 | 70- | |
| | ATC GAT AGA TGG AAA TAC CAA | | | | | | | dhfr(15)620 | |
| | TAC ATT CTC ATC ACT GGA AGT | | | | | | | | |
| | GAA GCT T (SEQ ID NO:28) | | | | | | | | |
| suII | AGC GCC GGC GGG GTC TAG CCG | 70 | 960-891 | 82.5 | 62.9 | Tn2l, Integron | X12869 | 70-sul | |
| | GCG GCT CTC ATC GAA GAA GGA | | | | | class | | (1)960 | |
| | GTC CTC GGT GAG ATT CAG AAT | | | | | 1, sulI | | | |
| | GCC GAA C (SEQ ID NO:29) | | | | | | | | |
| suIII | TAC GCG CCT GCG CAA TGG CTG | 70 | 420-351 | 82.8 | 61.4 | RSF1010, sulII | M36657 | 70-sul(2)420 | |
| | CGT CTG GCG CCA GAT ACC GGC | | | | | | | | |
| | CTC CAT CGG AGA AAC TGT CCG | | | | | | | | |
| | AGG TTA T (SEQ ID NO:30) | | | | | | | | |
| integron | TTG GAT GCC CGA GGC ATA GAC | 70 | 1200- | 78.3 | 51.4 | integrase, Int1 | M33633 | 70 | |
| classe 1 | TGT ACC CCA AAA AAC AGT CAT | | 1131 | | | | | int(1)1200 | |
| 3″-CS | AAC AAG CCA TGA AAA CCG CCA | | | | | | | | |
| | CTG CGC C (SEQ ID NO:31) | | | | | | | | |
| Gram | | | | | | | | | |
| postive | | | | | | | | | |
| vat | TTT ACC GAT AAA AGG GAA TCG | 70 | 2822- | 68.3 | 31.4 | AF117258 | 70-vat2822 | | |
| | GAA TCT TCA ATT TAT AAA ACC | | 2753 | | | | | | |
| | TAC TAT AAC GAA CGA AAA CAT | | | | | | | | |
| | TTT GGT G (SEQ ID NO:32) | | | | | | | | |
| vatC | GAA CAT GTT TAT TAC CTT CTA | 70 | 1376- | 71 | 37.1 | AF015628 | 70- | | |
| | TAG GGT ATA TTT CTT CTG GAT | | 1307 | | | | vatC1376 | | |
| | TGG GGC CTT GCT GAT TTT GCC | | | | | | | | |
| | ATT TCA T (SEQ ID NO:33) | | | | | | | | |
| vatD | TTG ATC TAA TTT TGG CAT ATG | 70 | 3022- | 69.2 | 32.9 | AF368302 | 70- | | |
| | TTT CTC CCA TCC ATT ACC AAA | | 2953 | | | | vatD3022 | | |
| | TAA ATT AAA TGG ATA TGT TGA | | | | | | | | |
| | GCC ATC C (SEQ ID NO:34) | | | | | | | | |
| vatE | CGT TCT TGA TAA AGT CTA GCT | 70 | 70-1 | 71.7 | 41.4 | AY043213 | 70-vatE70 | | |
| | CTA TGA GGA TGA GGT TAG GAT | | | | | | | | |
| | AGA CTG CAT TTG CGT CAG GTA | | | | | | | | |
| | TAG TCA T (SEQ ID NO:35) | | | | | | | | |
| vga | GAG CTT CAA TTG AGG AAT AAG | 70 | 1133- | 69.1 | 31.4 | M90056 | 70-vga1133 | | |
| | TTC ACA ATG TGA AAA TTG TTT | | 1064 | | | | | | |
| | TAC AAT ACC TTC TTC AGG CAC | | | | | | | | |
| | AAT TTT T (SEQ ID NO:36) | | | | | | | | |
| vgb | CCA TAT GGT ATA ACC GTT TCA | 70 | 3720- | 68.8 | 32.9 | AF117258 | 70-Vgb3720 | | |
| | GAT AAG GGG AAA GTT TGG ATT | | 3651 | | | | | | |
| | ACA CAA CAT AAA GCA AAT ATG | | | | | | | | |
| | ATA AGT T (SEQ ID NO:37) | | | | | | | | |
| vgbB | CTG ATG AAG TTA TAC CGT ATG | 70 | 468-399 | 68.8 | 34.3 | AF015628 | 70-vgbB468 | | |
| | GAC CTG AAT CGG GAA TAG ACA | | | | | | | | |
| | AGT TAA ACT CCT CTA AAT AAA | | | | | | | | |
| | AAT TCA T (SEQ ID NO:38) | | | | | | | | |

The present invention will be more readily understood by referring to the following examples which are given to illustrate the invention rather than to limit its scope.

### EXAMPLE I

Taxonomic Identification of Microorganisms Present in a Commercial Consortium

The following experiment was conducted to establish the concept of the invention and obtain preliminary results. A DNA microarray slide (Corning Ultragaps, Corning, N.Y.) was printed with DNA sequences using conventional technique in the art for attaching on the slide a number of sequences of genes as detailed in Table 2 below.

TABLE 2
Interpretation key (probes for Biozyme 5000 in grey)

| | | | |
|---|---|---|---|
| + cont GFP | A. oryzae 18S | P. denitrificans nos Z | + cont GFP |
| − cont GFP | A. oryzae pepO | P. fluorescens 16S | − cont GFP |
| A. globiformis 23S | 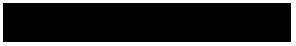 | R. eutropha 16S | A. Hydrophila aly |
| A. oxydans recA | 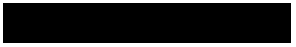 | S. cerevisiae 18S | A. salmonicida bhem1 |
| 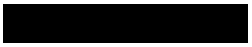 | B. megaterium cpn 60 | S. typhi dlt | A. globiformis 16S |
| C. jejuni gtpase | B. megaterium merR2 | S. scabies 16S | A. globiformis est |
| C. albicans MNT1 | 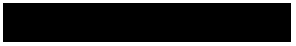 | S. elongatus 16S | A. oxydans 16S |
| E. coli stx2A | B. cepacia pvdA | 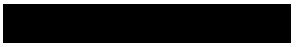 | A. niger calnexin |
| + cont A. thaliana | N. winogradskyi 16S | P. denitrificans nir S | C. jejuni gtpase |
| 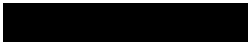 | N. europa amo A | 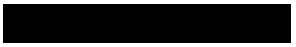 | C. albicans MNT1 |
| S. elongatus 16S | Nitrosomonas nir K | 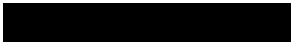 | C. parvum lax |
| Buffer | P. denitrificans nir S | P. denitrificans nos Z | E. faecalis 16S |
| Buffer | 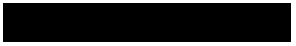 | R. eutropha gyrB | E. coli gus A |
| Buffer | 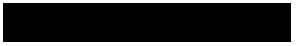 | S. scabies 16S | E. coli sltlle |
| Buffer | P. aeruginasa 16S | S. elongatus 16S | K. pneumoniae cpn 60 |
| Buffer | P. aeruginosa toxA | + cont A. thaliana | N. hambergensis nor B |

Table 3 lists the oligonucleotides probes immobilized on the microarray prototype used to analyze a commercial consortium; the layout of the microarray found in Table 2.

TABLE 2
Sequences of the probes for Biozyme 5000

| | | | Gen Bank | | | GC | |
|---|---|---|---|---|---|---|---|
| Probe | Organism | Gene | Accession no. | Start | Sequence | % | Tm |
| A. | A. oryzae | pepO | ASNPEPA | 721-770 | TTTCCAGAAGGCTTGTAGACGTCGTGGCCGTCTGCTCGGACTTGG | 64 | 84 |
| | | | | | GGAG (SEQ ID NO:39) | | |
| B. | A. globiformis | 23S | ARG23RRNAD | 11-60 | CACCCACAAGGGGTGTCAGGCAGGTCTCGGGCGGTTAGTATCCCC | 62 | 83 |
| | | | | | TGTTC (SEQ ID NO:40) | | |
| C. | A. oxydans | recA | AF214789 | 1-50 | TCCCAAAGCAACATCCAGGGCAATGGATCCGGTGGGGATGACCTC | 58 | 82 |
| | | | | | GATCG (SEQ ID NO:41) | | |
| D. | B. subtilis | 16S-23S | BSUB0005 | 144980- | GMCACGTTTCGMGGAATGATCCTTCAAAACTAAACAAGACAGGGA | 42 | 74 |
| | | | | 145029 | ACG (SEQ ID NO:42) | | |
| E. | A. salmonicida | bhem1 | AS17BHEM1 | 1490- | AGTCTCGTCACAGGTCACGGCGCTCAGGCCATGCTCGGCGCCGGC | 70 | 80 |
| | | | | 1539 | GCTCA (SEQ ID NO:43) | | |
| F. | s. typhi | dlt | STDLT | 661-710 | GAAGGCGGCATTGTTGATATGGTAACGGCCACGGACATACACGGA | 56 | 74 |
| | | | | | AGGCG (SEQ ID NO:44) | | |
| G. | P. denitrificans | nosZ | AF016059 | 1046- | TTCTCCGGGTGCAGCGGGCCGGTGGGCAGGAAGCGGTCCTTGGAG | 66 | 86 |
| | | | | 1095 | AACTT (SEQ ID NO:45) | | |
| H. | P. fluorescens | 16S | PSEIAM12 | 48-97 | CCGTCCGCCTCTCTCAAGAGAAGCAAGCTTCTCTCTACCGCTCGA | 58 | 81 |
| | | | | | CTTGC (SEQ ID NO:46) | | |
| I. | R. eutropha | 16S | AFARGSSA | 146-195 | CGCTTTCACCCTCAGGTCGTATGCGGTATTAGCTAATCTTTCGAC | 46 | 70 |
| | | | | | TAGTT (SEQ ID NO:47) | | |
| J. | A. globiformis | 16S | AGRDNA16 | 66-115 | GGGCAGGTTACTCACGTGTTACTCACCCGTTCGCCACTAATCCCC | 60 | 82 |
| | | | | | GGTGC (SEQ ID NO:48) | | |
| K. | S. scabies | 16S | AB026210 | 67-116 | CGTGTTACTCACCCGTTCGCCACTAATCCCCACCGAAGTGGTTCA | 54 | 74 |
| | | | | | TCGTT (SEQ ID NO:49) | | |
| L. | A. globiformis | esterase | E04386 | 51-100 | AGGCCGCGAGCTGGGCTGAATATTCCCGGTCTTCGCTCAGGAAAC | 62 | 84 |
| | | | | | GGCCA (SEQ ID NO:50) | | |
| M. | N. winogradskyi | 16S | NITRGDW | 46-95 | ACGCGTTACTCACCCGTCTGCCACTGACGTATTGCTACGCCCGTT | 58 | 82 |
| | | | | | CGACT (SEQ ID NO:51) | | |
| N. | P. polymyxa | 16S | AJ223989 | 66-115 | TTACTCACCCGTCCGCCGCTAGGCTTATATAGAAGCAAGCTTCTA | 48 | 71 |
| | | | | | CGATA (SEQ ID NO:52) | | |
| O. | P. polymyxa | 16S | AJ223989 | 66-115 | TTACTCACCCGTCCGCCGCTAGGCTTATATAGAAGCAAGCTTCTA | 48 | 71 |
| | | | | | CGATA (SEQ ID NO:53) | | |
| P. | S. elongatus | 16S | AF410931 | 206-255 | TGCTCCGTCAGGCTTTCGCCCATTGCGGAAAATTCCCCACTGCTG | 60 | 84 |
| | | | | | CCTCC (SEQ ID NO:54) | | |
| Q. | S. elongatus | 16S | AF410931 | 206-255 | TGCTCCGTCAGGCTTTCGCCCATTGCGGAAAATTCCCCACTGCTG | 60 | 84 |
| | | | | | CCTCC (SEQ ID NO:55) | | |
| R. | Eubacterial | 16S | ECRRNBZ | 325-374 | TTGTGCAATATTCCCCACTGCTGCCTCCCGTAGGAGTCTGGACCG | 56 | 81 |
| | | | | | TGTCT (SEQ ID NO:56) | | |
| S. | P. aeruginosa | 16S | AB037563 | 52-101 | TCACCCGTCCGCCGCTGAATCCAGGAGCAAGCTCCCTTCATCCGC | 64 | 54 |
| | | | | | TCGAC (SEQ ID NO:57) | | |
| T. | P. aeruginosa | toxA | AF227421 | 121-170 | GAAGGTGCCGTGGTAGCCGACGAACACATAGCCGCGCTCCTCCAG | 64 | 84 |
| | | | | | TTGGC (SEQ ID NO:58) | | |
| U. | R.eutropha | gyrB | A6014982 | 51-100 | CTGTGGATGGTGACCTGGATCTCGGTGCAGTAGCCGGCCAGCGCT | 64 | 84 |
| | | | | | TCGTC (SEQ ID NO:59) | | |
| V. | P. denitrificans | nosZ | AF016059 | 860-909 | TCTTCCAGGTTCCATTTGACCAGCTGGCTGTCGATGAACAGCGTG | 52 | 73 |
| | | | | | GTGTA (SEQ ID NO:60) | | |
| W. | N. hamburgensis | norB | NHNORB | 821-870 | CCAGTTGAAGTAGGTCTTCTTGTACGGGCAGCCGGAGACGCACAT | 60 | 82 |
| | | | | | GCGCC (SEQ ID NO:61) | | |
| X. | C. albicans | MNT | CAMNT1PRT | 121-170 | CCAGCAGCAACATTACCGGTCTGTTTTTCATGAGCGGCGGGTGAT | 50 | 80 |
| | | | | | TGTGT (SEQ ID NO:62) | | |
| Y. | Negative control | GFP. | AEVGFP | 595-644 | GGCCTAGAGGGTCCTGTTCGCAGGTGATAAAAGGATGAGGGAAAT | 52 | 73 |
| | 1 | | | | GTCGT (SEQ ID NO:63) | | |
| Z. | Negative control | GFP. | AEVGFP | 371-420 | ACACACCTAACTAGTAAACGTTTAATTTCAATCTTTTGCACATCA | 30 | 64 |
| | 2 | | | | TAGTT (SEQ ID NO:64) | | |

The DNA microarray slide was then hybridized overnight at 42° C. for 16 hours with 500 ng of Biozyme 5000 (Mirus B (6 Sep. 2002)) DNA. The DNA had previously been labeled with Cy3 16% in DIG hyb buffer: The hybridization volume was 6 μl on a cover slip of dimension 11 mm×11 mm. After hybridization, the cover slip is removed in 1×SC at room temperature followed by three washes. The first wash is made in 1xSSC, 0.2% SDS at 37° C. for 10 minutes. The second wash is made in 0.1xSSC, 0.2% SDS at room temperature for 5 minutes. Finally the third wash is made in 0.1xSSC, at room temperature for 5 minutes.

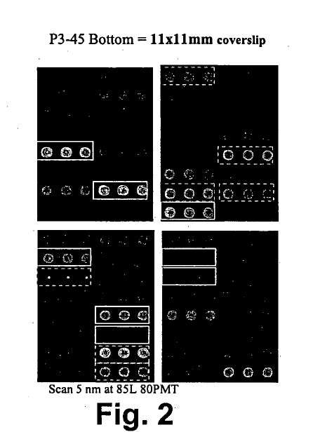

As a result, the interpretation key for the triplicate probes (see Table 2) identifies which spots represent which genes. Probes for any of the three bacterial species claimed to be present in the Biozyme 5000 consortia (B. subtilis, B. licheniformis, and P. polymyxa)are highlighted in grey. The strong signals were obtained from the expected microorganism Bacillus subtilis (see FIG. 2).

### EXAMPLE II

### Discriminating Power of cpn60 Probes Between Two Bacilleaceae Species

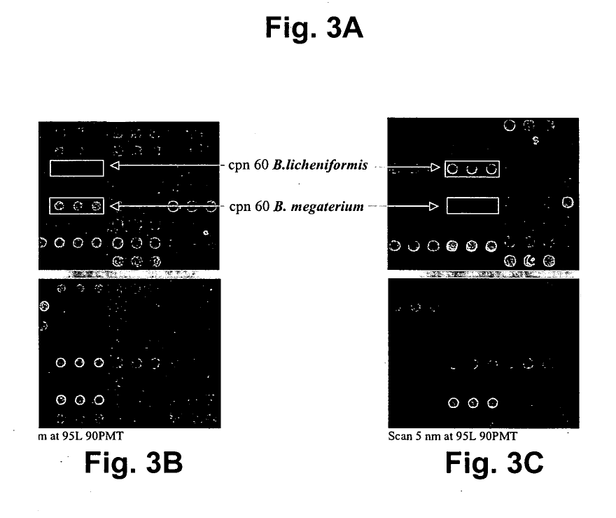

A microarray plate as in example I above with the same array layout and probe sequences is being used herein to illustration the superior specificity of cpn60 probes compared to 16S probes. The left panel (FIG. 3A) shows fluorescent labelled DNA from B. megaterium applied to array. The right panel (FIG. 3B) shows fluorescently labelled DNA from B licheniformis applied to array. The results obtained are illustrated in FIGS. 3A and 3B. As can be seen in FIGS. 3A and 3B, the cpn60 probe specific for B. licheniformis gives a signal when hybridized with B. licheniformis genomic DNA, but not at all with B. megaterium genomic DNA and vice versa (upper panels). This is not the case with the 16S probes (lower panels) that seem to light up much more easily and cross react with other 16S probes for different species. This results demonstrates the extra resolving power of cpn60 probes

### EXAMPLE III

### Microarray Using 16S and cpn60 Amplicons

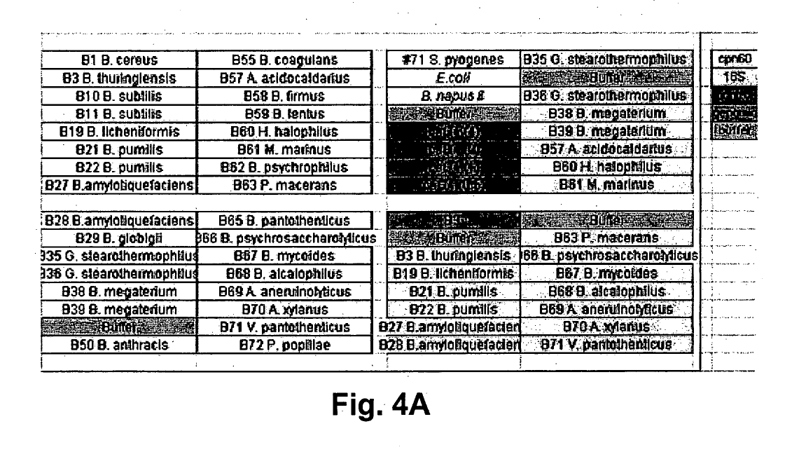

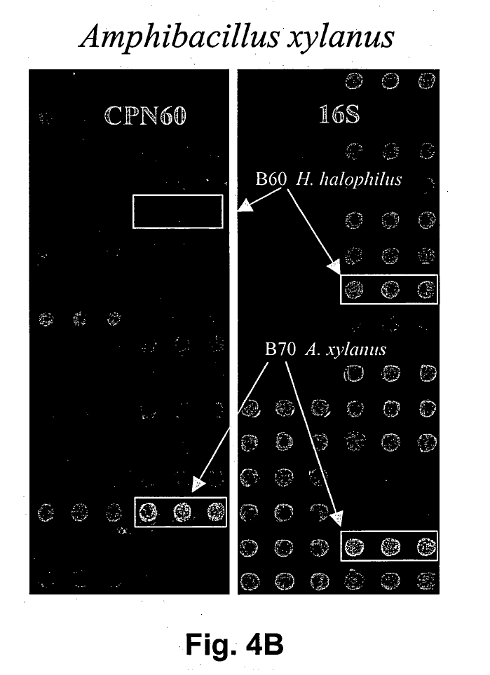

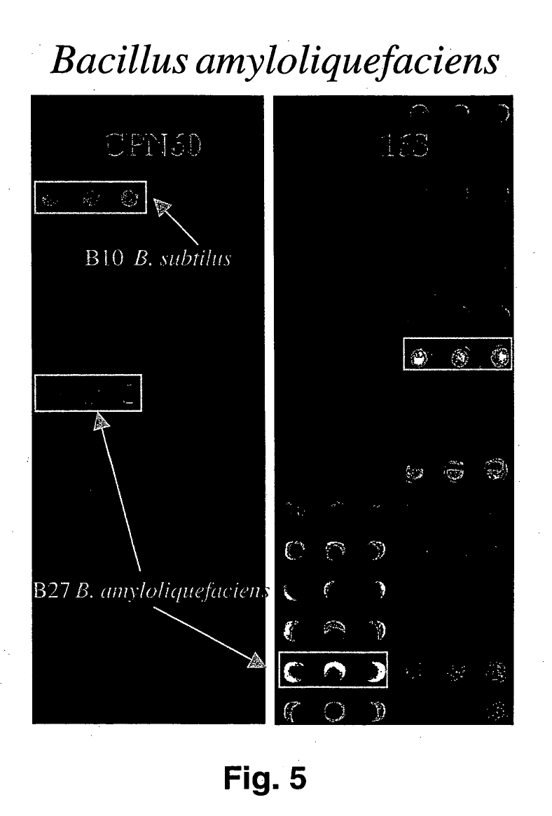

A microarray plate was printed with the following sequences found in Table 3 using the key found in FIG. 4A.

TABLE 3
SEQUENCES USED FOR AMPLICON ARRAY

| | | GenBank | | |
|---|---|---|---|---|
| Organism | Gene | Accession no. | Sequence | |
| subtilis | 16S | ATCC 9799 | TGTTAGGGAAGAACAAGTGCCGTTCAAATAGGGCGGCACCTTGACGGTACCTAACCAGAAAGCCACGGCT | +TL,64 |
| | | | AACTACGTGCCAGCAGCCGCGGTAATACGTAGGTGGCAAGCGTTGTCCGGAATTATTGGGCGTAAACGGC | |
| | | | TCGCAGGCGCTTTCTTAAGTCTGATGTGAAAGCCCCCGGCTCAACCGGGGAGGGTCATTGGAAACTGCGG | |
| | | | AACTTGAGTGCAGAAGAGGAGAGTGGAATTCCACGTGTAGCGGTGAAATGCGTAGAGATGTGGAGGAACA | |
| | | | CCAGTGGCGAAGGCGACTCTCTGGTCTGTAACTGACGCTGAGGAGCGAAAOCGTGGGGAGCGAACAGGAT | |
| | | | TAGATACCCTGGTAGTCCACGCCGTAAACGATGAGTGCTAAGTGTTAGGGGGTTTCCGCCCCTTAGTGCT | |
| | | | GCAGCTAACGCATTAAGCACTCCGCCTGGGGAGTACGGTCGCAAGACTGAAACTCAAAGGAATTGACGGG | |
| | | | GGCCCGCACAAGCGGTGGAGCATGTGGTTTAATTCGAAGCAACGCGAAGAACCTTACCACGTCTTGACAT | |
| | | | CCTCTGACAATCCTAGAGATAGGACGTCCCCTTCGGGGGCAGAGTGACAGGTGGTGCATGGTTGTCGTCA | |
| | | | GCTCGTGTCGTGAGATGTTGGGTTAAGTCCCGCAACGAGCGCAACCCTTGATCTTAGTTGCCAGCATTCA | |
| | | | GTTGGGCACTCTAAGGTGACTGCCGGTGACAAACCGGAGGAAGGTGGGGATGACGTCAAATCATCATGCC | |
| | | | CCTTATGACCTGGGCTACACACGTGCTACAATGGGCAGAACAAAGGGCAGCGAAACCGCGAGGTTAAGCC | |
| | | | AATCCCACAAATCTGTTCTCAGTTCGGATCGCAGTCTGCAACTCGACTGCGTGAAGCTGGAATCGCTAGT | |
| | | | AATCGCGGATCAGATGCCGGGTGATACGTTCCCGGGCCTTGTACACCGCCCGTCACACCACGAGAGTTTG | |
| | | | TAACACCCGAAGTCGGTGAGGTAACCTTTTTGGAGCCAGCCGCCGAAGGTGGGACAGATGATTGGGTTC | |
| | | | (SEQ ID NO: 65) | |
| Bacillus | 16S | DSM 13 | AGAGTTTGATCCTGGCTCAGGACGAACGCTGGCGGCGTGCCTAATACATGCAAGTCGAGCGGACCGACG | |
| licheniformis | | | GGAGCTTGCTCCCTTAGGTCAGCGGCGGACGGGTGAGTAACACGTGGGTAACCTGCCTGTAAGACTGGG | |
| | | | ATAACTCCGGGAAACCGGGGCTAATACCGGATGCTTGATTGAACCGCATGGTTCAATCATAAAAGGTGG | |
| | | | CTTTTAGCTACCACTTACAGATGGACCCGCCGCGCATTAGCTAGTTGGTGAGGTAACGGCTCACCAAGG | |
| | | | CGACGATGCGTAGCCGACCTGAGAGGGTGATCGGCCACACTGGGACTGAGACACGGCCCAGACTCCTAC | |
| | | | GGGAGGCAGCAGTAGGGAATCTTCCGCAATGGACGAAAGTCTGACGGAGCAACGCCGCGTGAGTGATGA | |
| | | | AGGTTTTCGGATCGTAAAACTCTGTTGTTAGGGAAGAACAAGTACCGTTCGAATAGGGCGGTACCTTGA | |
| | | | CGGTACCTAACCAGAAAGCCACGCCTAACTACGTGCCAGCAGCCGCGGTAATACGTAGGTGGCAAGCGT | |
| | | | TGTCCCGAATTATTGGGCGTAAAGCGCGCGCAGGCGGTTTCTTAAGTCTGATGTGAAAGCCCCCGGCTC | |
| | | | AACCGGGGAGGGTCATTGGAAACTCGGGAACTTGAGTGCAGAAGAGGACAGTGGAATTCCACGTGTAGC | |
| | | | CGTGAAATGCGTAGAGATGTCGAGGAACACCAGTGGCGAAGGCGACTCTCTGGTCTGTAACTGACGCTG | |
| | | | AGGCGCGAAAGCGTGGGGAGCGAACAGGATTAGATACCCTGGTAGTCCACGCCGTAAACGATGAGTGCT | |
| | | | AAGTGTTAGAGGGTTTCCGCCCTTTAGTGCTGCAGCAAACGCATTAAGCACTCCGCCTGGGGAGTACGG | |
| | | | TCGCAAGACTGAAACTCAAAGGAATTGACGCGGGCCCGCACAAGCGGTGGAGCATGTGGTTTAATTCGA | |
| | | | TAGAGATAGGGCTTCCCCTTCGGGCGCAGAGTGACAGGTGGTGCATGGTTGTCGTCAGCTCGTGTCGTG | |
| | | | AGATGTTGCGTTAAGTCCCGCAACGAGCGCAACCCTTGATCTTAGTTGCCAGCATTCAGTTGGGCACTC | |
| | | | TAAGGTGACTGCCGGTGACAAACCGGAGGAAGGTGGGGATGACGTCAAATCATCATGCCCCTTATGACC | |
| | | | TGGGCTACACACGTGCTACAATGGGCAGAACAAAGGGCAGCGAAGCCGCGAGGCTAAGCCAATCCCACA | |
| | | | AATCTGTTCTCAGTTCGGATCGCAGTCTGCAACTCGACTGCGTGAAGCTGGAATCGCTAGTAATCGCGG | |
| | | | ATCAGCATGCCGCGGTGAATACGTTCCCGGGCCTTCTACACACCGCCCGTCACACCACGAGAGTTTGTA | |
| | | | ACACCCGAAGTCGGTGAGGTAACCTTTTGGAGCCAGCCGCCGAAGGTGGGACAGATGATTGGGGTGAAG | |
| | | | TCGTAACAAGGTAGCCGTATCGGAAGGTGCGGCTGGATCACCTCCTTTCT (SEQ ID NO:66) | |
| Bacillus | 16S | NCDO | GAGAGTTTGATCCTGGCTCAGGACGAACGCTGGCGGCGTGCCTAATACATGCAAGTCGAGCGGACAGAA | |
| pumilus | | 1766 | GGGAGCTTGCTCCCGGATGTTAGCGGCGGACGGGTGAGTAACNCGTGGGTAACCTNCCTGTNAGACTGG | |
| | | | GATAACTCCGGGAAACCGGAGCTAATACCGGATAGTTCCTTGAACCGCATNGTACAAGGATGAAAGACC | |
| | | | GTNTCGGCTATCACTTACAGATNGACCCGCGGCCCATTAGCTAGTTGGTGGGGTAATGGCTCACCAAGG | |
| | | | CGACGATGCGTAGCCGACCTGAGAGGGTNATCGGCCACACTGGGACTGAGACACGGCCNNGACTCCTAC | |
| | | | GGGAGGCAGCAGTAGGGAATCTTCCGCAATGGACGAAAGTCTGACGGAGCAACGCCGCGTGAGTGATGA | |
| | | | AGGTTTTCGGATCGTNAAGCTCTGTTGTTAGGGAAGAACAAGTGCGAGAGTAACTNCTCGCACCTTGAC | |
| | | | GGTACCTAACCAGAAAGCCACGGCTAACTACGTGCCAGCAGCCGCGGTNATACGTAGGTGGCAAGCGTT | |
| | | | GTCCGGAATTATTGGGCGTNAAGGGCTCGCAGGCGGTTTCTTAAGTCTNATGTGAAAGCCCNCNGCTCA | |
| | | | ACCGGGGAGGGTCATTGGAAACTGGGNAACTTGAGTGCAGAAGAGGAGAGTGGAATTCCACGTGTAGCG | |
| | | | GTNAAATGCGTAGAGATGTGGAGGAACACCAGTGGCGAAGGCGACTCTCTNGTCTGTAACTNACGCTGA | |
| | | | GGAGCGAAAGCGTGGGGAGCGAACAGGATTAGATACCCTGGTAGTCCACGCCGTAAACGATGAGTGCTN | |
| | | | AGTGTTAGGGGGTTTCCGCCCCTTAGTGCTNCANCTAACGCATTAAGCACTCCGCCTGGGGAGTACGGT | |
| | | | CGCAAGACTNAAACTCAAAGGAATTGACGGGGGCCNGCACAAGCGGTGGAGCATGTNGTTTAATTCGAA | |
| | | | GNAACGCGAAGAACCTTACCAGGTCTTGACATCCTCTGACAACCCTAGAGATAGGGNTNTCTTCGGGGA | |
| | | | CAGAGTGACAGGTGGNGCATNGTNGTCGTCAGCTCGTGTCGTGAGATGTTGGOTTAAGTCCCGCAACGA | |
| | | | GCGCAACCCTTGATCTTAGTTGCCAGCATTTAGTTGGGCACTCTAAGGTGACTGCCGGTGACAAACCGG | |
| | | | AGGAAGGTNGGGATGACGTCAAATCATCATGCCCCTTATGACCTNGGCTACACACGTGCTACAATGGAC | |
| | | | AGACNAAGGGCTGCGAGACCGCAAGGTTTAGCCAATCCCATAAATCTGTTCTCAGTTCGGATCGCAGTC | |
| | | | TGCNACTNGACTGCGTGAAGCTGGAATCGCTAGTAATCGCGGATCGCATOCCGCGGTGAATACGTTC | |
| | | | CCGGGCCTNGTACACACCGCCCGTCACACCACGAGAGTTTGNAACACCC (SEQ ID NO:67) | |
| B. amylolique- | 16S | ATCC | GAGAGTTTGATCCTGGCTCAGGACGAACGCTGGCGGCGTGCCTAATACATGCAAGTCGAGCGGACAGAT | |
| faciens | | 23350 | GGGAGCTTGCTCCCTGATGTTAGCGGCGGACGGGTGAGTAACACGTGGGTAACCTGCCTGTAAGACTGG | |
| | | | GATAACTCCGGGAAACCGGGGCTAATACCGGATGCTTGTTTGAACCGCATGGTTCAACATAAAGGTGGC | |
| | | | TTCGGCTACCACTTACAGATGGACCCGCGGCGCATTAGCTAGTTGGTGAGGTAACGGCTCACCAAGGCG | |
| | | | ACGATGCGTAGCCGACCTGAGAGGGTGATCGGCCACACTGGGACTGAGACACGGCCCAGACTCCTACGG | |
| | | | GAGGCAGCAGTAGGGAATCTTCCGCAATGGACGAAAGTCTGACGGAGCAACGCCGCGTGAGTGATGAAG | |
| | | | GTTTTCGGATCGTAAAGCTNTGTTGTTAGGGAAGAACAAGTGCCGTTCAAATAGGGCGGNACCTNGACG | |
| | | | GTACCTAACCAGAAAGCCACGGCTAACTACGTGCCAGCAGCCGCGGTNATACGTAGGTGGCNAGCGTTG | |
| | | | TCCGGAATTNTTGGGCGTNAAGGGCTCGCAGGCGGTTTCTTNAGTCTGATGTGAAAGCCCCCGGCTCAA | |
| | | | CCGGGGAGGGTCATTGGAAACTGGGGAACTTGAGTGCAGAAGAGGAGAGTGGAATTCCACGTGTAGCGG | |
| | | | TGAAATGCGTAGAGATGTGGAGGAACACCAGTGGCGAAGGCGACTCTCTGGTTGTAACTGACGCGAGGA | |
| | | | GCGAAGCGTGGGGAGCGAACAGGATTAGATACCCTGGTAGTCCACGCCGTAAACGATGAGTGCTAAGTG | |
| | | | TTAGGGGGTTTCCGCCCCTTAGTGCTGCAGTAACGCATTAAGCACTCCGCCTGGGGAGTACGGTCGCAA | |
| | | | GACTNAAACTCAAAGGAATTGACGGGGCCGCACAAGCGGTGGAGCATGTGGTTTAATTCGAAGCAACGC | |
| | | | GAAGAACCTTACCAGGTCTTGACATCCTCTGACAATCCTAGAGATAGGACGTCTTCGGGGGCAGAGTGA | |
| | | | ACAGGTGGTGCATGGTTGTCGTCAGCTCGTGTCGTGAGATGTTGGGTTAAGTCCCGCAACGAGCGCAAC | |
| | | | CCTTGATCTTAGTTGCCAGCATTCAGTTGGGCACTCTAAGGTGACTGCCGGTGACAAACCGGAGGAAGG | |
| | | | TGGGGATGACGTCAAATCATCATGCCCCTTATGACCTNGGCTACACACGTGCTACNATGGGCAGAACNA | |
| | | | AGGGCAGCGAAACCGCGAGGTCAAGCCAATCCCACAAATCTATTCTCAGTTCGGATCGCAGTCTGCAAC | |
| | | | TCGACTGCGTGAAGCTGGAATCGCTAGTAATCGCGGATCAGCATGCCGCGGTGAATACGTTCCCGGGCC | |
| | | | TTGTACACACCGCCCGTCACACCACGAGAGTTTGTAACACCC (SEQ ID NO:68) | |
| Bacillus | 16S | IAM | GATGAACGCTGGCGGCGTGCCTAATACATGCAAGTCGAGCGAATGGATTAAGAGCTTGCTCTTATGAAG | |
| cereus | | 12605 | TTAGCGGCGGACGGGTGAGTAACACGTGGGTAACCTGCCCATAAGACTGGGATAACTCCGGGAAACCGG | |
| | | | GGCTAATACCGGATAACATTTTGAACCGCATGGTTCGAAATTGAAAGGCGGCTTCGGCTGTCACTTATG | |
| | | | GATGGACCCGCGTCGCATTAGCTAGTTGGTGAGGTAACGGGCTCACCAAGGCAACGATGCGTAGCCGAC | |
| | | | CTGAGAGGGTGATCGGCCACACTGGGACTGAGACACGGCCCAGACTCCTACGGGAGGCAGCAGTAGGGA | |
| | | | ATCTTCCGCAATGGACGAAAGTCTGACGGAGCAACGCCGCGTGAGTGATGAAGGCTTTCGGGTCGTAAA | |
| | | | ACTCTGTTGTTAGGGAAGAACAAGTGCTAGTTGAATAAGCTGGCACCTTGACGGTACCTAACCAGAAAG | |
| | | | CACGGCTAACTACGTGCCAGCAGCCGCGGTAATACGTAGGTGGCAAGCGTTATCCGGAATTATTGGGCG | |
| | | | TAAAGCGCGCGCAGGTGGTTTCTTAAGTCTGATGTGAAAGCCCACGGCTCAACCGTGGAGGGTCATTGG | |
| | | | AAACTGGGAGACTTGAGTGCAGAAGAGGAAGTGGATTCCATGTGTAGCGGTGAAATGCGTAGAGATATG | |
| | | | GAGGAACACCAGTGGCGAAGGCGACTTTCTGGTCTGTAACTGACACTGAGGCGCGAAAGCGTGGGGAGC | |
| | | | AAACAGGATTAGATACCCTGGTAGTCCACGCCGTAAACGATGAGTGCTAAGTGTTAGAGGGTTTCCGCC | |
| | | | CTTTAGTGCTGAAGTTAACGCATTAAGCACTCCGCCTGGGGAGTACGGCCGCAAGGCTGAAACTCAAAG | |
| | | | GAATTGACGGGGGCCCGCACAAGCGGTGGAGCATGTGGTTTAATTCGAAGCAACGCGAAGAACCTTACC | |
| | | | AGGTCTTGACATCCTCTGAAAACCCTAGAGATAGGGCTTCTCCTTCGGGAGCAGAGTGACAGGTGGTGC | |
| | | | ATGGTTGTCGTCAGCTCGTGTCGTGAGATGTTGGGTTAAGTCCCGCAACGAGCGCAACCCTTGATCTTA | |
| | | | GTTGCCATCATTAAGTTGGGCACTCTAAGGTGACTGCCGGTGACAAACCGGAGGAGGTGGGGATGACGT | |
| | | | CAAATCATCAGTGCCCCTTATGACCTGGGCTACACACGTGCTACAATGGACGGTACAAAGAGCTGCAAG | |
| | | | ACCGCGAGGTGGAGCTAATCTCATAAAACCGTTCTCAGTTCGGATTGTAGCTGCAACTCGCCTACATGA | |
| | | | AGCTGGATCGCTAGTAATCGCGGATCAGATGCCGCGGTGATACGTTCCCGGCCTTGTACACACCGCCCG | |
| | | | TCACACCACGAGAGTTTGTAACACCCGAGTCGGTGGGGTAACCTTTTTGGAGCCAGCCGCCTAAGGTGG | |
| | | | GACAGATGATTGGGGTGAAGTCGTAACAA (SEQ ID NO:69) | |
| Bacillus | 16S | NUB3621 | GCGGCGTGCCTAATACATGCAGTCGAGCGGACTCGCGGCGAGCTTGCTTTGCCTTGGTCAGCGGCGGAC | |
| stearothermo- | | | GGGTGAGTAACACGTGGGTAACCTGCCCGCAAGACCGGGATAACTCCGGGAACCGGGGCTAATACCGGA | |
| philus | | | TAACACCGAGACCGCATGGTCTTCGGTTGAAAGGCGGCTTCGGCTGCCACTTACTGATGGGCCCGCGGC | |
| | | | GCATTAGCTAGTTGGTGAGGTAACGGCTCACCAAGGCGACGATGCGTAGCCGGCCTGAGAGGGTGACCG | |
| | | | CACACTGGGACTGAGACACGGCCCAGACTCCTACGGGAGGCAGCAGTAGGAATCTTCCCAATGGACGAA | |
| | | | AGTCTGACGGAGCGACGCCGCGTGAGCGAAGAAGGCCTTCGGGACGTAAAGCTCTGTTGTTAGGGAAGA | |
| | | | AGAAGTGCCGTTCGAACAGGGCGGTCCGGTGAACGTACCTACCGAGAAAGCCCCGGCTAACTACGTGCC | |
| | | | AGCAGCCGCGGTAATACGTAGGGGGCGAGCGTTGTCCGGAATTATTGGGCGTAAAGCGCGCGCAGGCGG | |
| | | | TCCCTTAAGTCTGATGTGAAAGCCCACGGCTTAACCGTGGAGGGTCATTGGAAACTGGGGGACTTGAGT | |
| | | | GCAGGAGAGGAGACGGAATTCCACGTGTAGCGGTGAAATGCGTAGAGATGTGGAGGAACACCAGTGGCG | |
| | | | AAGGCGGCTCTCTGGTCCGTCTCTGACGCTGAGGCGCGAAAGCGTGGGGAGCAAACAGGATTAGATACC | |
| | | | CTGGTAGTCCACGCCGTAAACGATGAGTGCTAAGTGTTAGAGGGGTATTCCCTTTAGTGCTGTATCTAA | |
| | | | CGCGTTAAGCACTCCGCCTGGGGAGTACGGCCGCAAGGCTGAAACTCAAAGGAATTGACGGGGGCCCGC | |
| | | | ACAAGCGGTGGAGCATGTGGTTTAATTCGAAGCAACGCGAAGAACCTTACCAGGTCTTGACATCCCCTG | |
| | | | ACAACCCTGGAGACAGGGCGTTCCCCCCTTGCGGGGACAGGGTGACAGGTGGTGCATGGTTGTCGTCAG | |
| | | | CTCGTGTCGTGAGATGTTGGGTTAAGTCCCGCAAGCGCAACCCTCGCCCCTAGTTGCCAGCATTCATTT | |
| | | | GGGCACTCTAGGGGGACTGCCGGCTAAAACTCAGAGGAAGGTGGGGATGACGTCAAATCATCATGCCCC | |
| | | | TTATGACCTGGGCTACACACGTGCTACAATGGGCGGTACAAAGGGCTGCGAACCCGCGAGGGGGAGCGA | |
| | | | ATCCCAAAAAGCCGCTCTCAGTTCGGATTGCAGGCTGCAACTCGCCTGCATGAAGCCGGAATCGCTAGT | |
| | | | AATCGCGATCAGCATGCCCGGTGAATACGTTCCCGGGCCTTGTACACACCGCCCGTCACACCACGAGAG | |
| | | | CTTGCAACACCC (SEQ ID NO:70) | |
| Bacillus | 16S | IAM | GATGAACGCTGGCGGCGTGCCTAATACATGCAAGTCGAGCGAACTGATTAGAAGCTTGCTTCTATGACG | |
| megaterium | | 13418 | TTAGCGGCGGACGGGTGAGTAACACGTGGGCAACCTGCCTGTAAGACTGGGATAACTTCGGGAAACCGA | |
| | | | GGCTAATACCGGATAGGATCTTCTCCTTCATGGGAGATGATTGAAAGATGGTTTCGGCTATCACTTACA | |
| | | | GATGGCCCGGGTGCATTAGCTAGTTGGTGAGGTAACGGCTCACCAAGGCACGATGCGTAGCCGACCTGA | |
| | | | GAGGGTGATCGGCCACACTGGGACTGAGACACGGCCCAGACTCCTACGGGAGGCAGCAGTAGGGAATCT | |
| | | | TCCGCAATGGACGAAACTCTGACGGAGCAACGCCGCGTGAGTGATGAAGGCTTTCGGGTCGTAAAACTC | |
| | | | TGTTGTTAGGGAAGAACAAGTACAAGAGTAACTGCTTGTACCTTGACGGTACCTAACCAGAAAGCCACG | |
| | | | GCTAACTACGTGCCAGCAGCCGAAAGCCCACGGCTCAACCGTGGAGGGTCATTGGAAACTGGGGAACTT | |
| | | | GAGTGCAGAAGAGAAAAGCGGAATTCCACGTGTAGCGGTGAAATGCGTAGAGATGTGGAGGAACACCAG | |
| | | | TGGCGAAGGCGGCTTTTTGGTCTGTAACTGACGCTGAGGCGCGAAAGCGTGGGGAGCAAACAGGATTAG | |
| | | | ATACCCTGGTAGTCCACGCCGTAAACGATGAGTGCTAAGTGTTAGAGGGTTTCCGCCCTTTAGTGCTGC | |
| | | | AGCTAACGCATTAAGCACTCCGCCTGGGGAGTACGGTCGCAAGACTGAAACTCAAAGGAATTGACGGGG | |
| | | | GCCCGCACAAGCGGTGGAGCATGTGGTTTAATTCGAAGCAACGCGAAGAACCTTACCAGGTCTTGACAT | |
| | | | CCTCTGACAACTCTAGAGATAGAGCGTTCCCCTTCGGGGGACAGAGTGACAGGTGGTGCATGGTTGTCG | |
| | | | TCAGCTCGTGTCGTGAGATGTTGGGTTAAGTCCCGCAACGAGCGCAACCCTTGATCTTAGTTGCCAGCA | |
| | | | TTTAGTTGGTGCACTTAAGGTGACTGCCGGTGACAAACCGGAGGAAGGTGGGGATGACGTCAAATCATC | |
| | | | ATGCCCCTTATGACCTGGGCTACACACGTGCTACAATGGATGGTACAAGGGCTGCAAGACCGCGAGGTC | |
| | | | AAGCCAATCCCATAAAACCATTCTCAGTTCGGATTGTAGGCTGCAACTCGCCTACATGAAGCTGGAATC | |
| | | | GCTAGTAATCGCGGATCAGCATGCCGCGGTGAATACGTTCCCGGGCCTTGTACACACCGCCCGTCACAC | |
| | | | CACGAGAGTTTGTAACACCCGAAGTCGGTGGAGTAACCGTAAGGACGTAGCCGCCTAAGGTGOGACAGA | |
| | | | TGATTGGGGTGAAGTCGTAACAA (SEQ ID NO:71) | |
| Bacillus | 16S | IAM | GACGAACGCTGGCGGCGTGCCTAATACATGCAAGTCGTGCGGACCTTTTAAAAGCTTGCTTTTAAAAGG | |
| coagulans | | 12463 | TTAGCGGCGGACGGGTGAGTAACACGTGGGCAACCTGCCTGTAAGACNGGGATAACGCCGGGAAACCG | |
| | | | GGGCTAATACCNGATAGTTTTTTCCTCCGCATGGAGGAAAAAGGAAAGGCGGCTTCGGCTGCCACTTAC | |
| | | | AGATGGGCCCGCGGCGCATTAGCTAGTTGGCGGGGTAACGGCCCACCAAGGCAACGATGCGTAGCCGAC | |
| | | | CTGAGAGGGTGATCGGCCACATTGGGACTGAGACACGGCCCAAACTCCTACGGGAGGCAGCAGTAGGGA | |
| | | | ATCTTCCGCAATGGACGAAAGTCTGACGGAGCAACGCCGCGTGAGTGAAGAAGGCCTTCGGGTCGTAAA | |
| | | | ACTCTGTTGCCGGGGAAGAACAAGTGCCGTTCGAACAGGGCGGCGCCTTGACGGTACCCGGCCAGAAAG | |
| | | | CCACGGCTAACTACGTGCCAGCAGCCGCGGTAATACGTAGGTGGCAAGCGTTGTCCGGAATTATTGGGC | |
| | | | GTAAAGCGCGCGCAGGCGGCTTCTTAAGTCTGATGTGAAATCTTGCGGCTCAACCAAGCGGTCATTGGA | |
| | | | AACTGGGAGGCTTGAGTGCAGAAGAGGAGAGTGGAATTCCACGTGTAGCGGTGAAATGCGTAGAGATGT | |
| | | | GGAGGAACACCAGTGGCGAAGGCGGCTCTCTGGTCTGTAACTGACGCTGAGGCGCGAAAGCGTGGGGAG | |
| | | | CAAACAGGATTAGATACCCTGGTAGTCCACGCCGTAAACGATGAGTGCTAAGTGTTAGAGGGTTTCCGC | |
| | | | CCTTTAGTGCTGCACTAACGCATTAAGCACTCCGCCTGGGGAGTACGGCCGCAAGGCTGAAACTCAAAG | |
| | | | GAATTGACGGGGGCCGCACAAGCGGTGGAGCATGTGGTTTAATTCGAAGCAACGCGAAGAACCTTACCA | |
| | | | GGTCTTGACATCCTCTGACCTCCCTGGAGACAGGGCCTTCTTCGGGGGACAGAGTGACAGGTGGTGCAT | |
| | | | GGTTGTCGTCAGCTCGTGTCGTGAGATGTTGGGTTAAGTCCCGCAACGAGCGCAACCCTTGACCTTAGT | |
| | | | TGCCAGCATTGAGTTGGGCACTCTAAGGTGACTGCCGGTGACAAACCGGAGGAAGGTGGGGATGACGTC | |
| | | | AAATCATCATGCCCCTTATGACCTGGGCTACACACGTGCTACAATGGATGGTACAAAGGGCTGCGAGAC | |
| | | | CGCGAGGTTAAGCCAATCCCAGAAAACCATTCCCAGTTCGGATTGCAGGCTGCAACCCGCCTGCATGAA | |
| | | | GCCGGAATCGCTAGTAATCGCGGATCAGCATGCCGCGGTGAATACGTTCCCGGGCCTTGTACACACCGC | |
| | | | CCGTCACACCACGAGAGTTTGTAACACCCGAAGTCGGTGAGGTAACCTTTANGGAGCCAGCCGCCGAAG | |
| | | | GTGGGACAGATGATTGGGGTGAAGTCGTAACAA (SEQ ID NO:72) | |
| Alicycloba- | 16S | DSM 446 | CCTGGCTCAGGACGAACGCTGGCGGCGTGCCTAATACATGCAAGTCGAGCGGACTTTTCGGAGGTCAGC | |
| cillus | | | GGCGGACGGGTGAGGAACACGTGGGTAATCTGCCTTTCAGACCGGAATAACGCCCGGAAACGGGCGCTA | |
| acidocaldarius | | | ATGCCGGATACGCCCGCGAGGAGGCATCTTCTTGCGGGGAAAGGCCCGATTGGGCCGCTGAGAGAGGAG | |
| | | | CCCGCGGCGCATTAGCTGGTTGGCGGGGTAACGGCCCACCAAGGCGACGATGCGTAGCCGACCTGAGAG | |
| | | | GGTGACCGGCCACACTGGGACTGAGACACGGCCCAGACTCCTACGGGAGGCAGCAGTAGGGAATCTTCC | |
| | | | GCAATGGGCGCAAGCCTGACGGAAGCAACGCCGCGTGAGCGAAGAAGGCCTTCGGGTTGTAAAGCTCTG | |
| | | | TTGCTCGGGGAGAGCGGCATGGGGAGTGGAAAGCCCCATGCGAGACG | |
| | | | GTACCGAGTGAGGAAGCCCCGGCTAACTACGTGCCAGCAGCCGCGGTAAAACGTAGGGGGCGAGCGTTG | |
| | | | TCCGGAATCACTGGGCGTAAAGGGTGCGTAGGCGGTCGAGCAAGTCTGGAGTGAAAGTCCATGGCTCAA | |
| | | | CCATGGGATGGCTCTGGAAACTGCTTGACTTCAGTGCTGGAGAGGCAAGGGGAATTCCACGTGTAGCGG | |
| | | | TGAAATGCGTAGAGATGTGGAGGAATACCTGTGGCGAAGGCGCCTTGCTGGACAGTGACTGACGCTGAG | |
| | | | GCACGAAAGCGTGGGGAGCAAACAGGATTAGATACCCTGGTAGTCCACGCCGTAAACGATGAGTGCTAG | |
| | | | GTGTTGGGGGGACACACCCCAGTGCCGAAGGAAACCCAATAAGCACTCCGCCTGGGGAGTACGGTCGCA | |
| | | | AGACTGAAACTCAAAGGAATTGACGGGGGCCCGCACAAGCAGTGGAGCATGTGGTTTAATTCGAAGCAA | |
| | | | CGCGAAGAACCTTACCAGGGCTTGACATCCCTCTGACCGGTGCAGAGATGCACCTTCCCTTCGGGGCAG | |
| | | | AGGAGACAGGTGGTGCATGGTTGTCGTCAGCTCGTGTCGTGAGATGTTGGGTTCAGTCCCGCAACGAGC | |
| | | | GCAACCCTTGACCTGTGTTACCAGCGCGTTGAGGCGGGGACTCACAGGTGACTGCCGGCGTAAGTCGGA | |
| | | | GGAAGGCGGGGATGACGTCAAATCATCATGCCCCTGATGTCCTGGGCTACACACGTGCTACAATGGGCG | |
| | | | GTACAAAGGGAGGCGAAGCCGCGAGGCGGAGCGAAACCCAAAAAGCCGCTCGTAGTTCGGATTGCAGGC | |
| | | | TGCAACTCGCCTGCATGAAGCCGGAATTGCTAGTAATCGCGGATCAGCATGCCGCGGTGAATACGTTCC | |
| | | | CGGGCCTTGTACACACCGCCCGTCACACCACGAGAGTCGOCAACACCCGAAGTCGGTGAGGTAACCCCG | |
| | | | GGAAGGCGGGGATGACGTCAAATCATCATGCCCCTGATGTCCTGGGTCGTAACAAGGTAGCCGTACCGG | |
| | | | AAGGTGCGGCTG (SEQ ID NO:73) | |
| Bacillus | 16S | NCIMB | AGAGTTTGATCCTGGCTCAGGACGAACGCTGGCGGCGTGCCTAATACATGCAAGTCGAGCGAATGGATG | |
| lentus | | 8773 | GGAGCTTGCTCCCAGAAGTTAGCGGCGGACGGGTGAGTAACACGTGGGCAACCTACCTGTAAGACTGGG | |
| | | | ATAACTTCGGGAAACCGGAGCTAATACCGGATAACTTCTTTCTTCTCCTGGAGAAAGGTTGAAAGACGG | |
| | | | CTTCGGCTGTCACTTACAGATGGGCCCGCGGCGCATTAGCTACTTGGTGAGGTAACGGCTCACCAAGGC | |
| | | | AACGATGCGTAGCCGACCTGAGAGGGTGATCGGCCACACTGGGACTGAGACACGGCCCAGACTCCTACG | |
| | | | GGAGGCAGCAGTAGGGAATCTTCCGCAATGGACGAAAGTCTGACGGAGCAACGCCGCGTGAGTGATGAA | |
| | | | GGTTTTCGGATCGTAAAACTCTGTTATCAGGGAAGAACAAGTATCGGAGTAACTGCCGGTACCTTGACG | |
| | | | GTACCTGACCAGAAAGCCACGGCTAACTACGTGCCAGCAGCCGCGGTAATACGTAGGTGGCAAGCGTTG | |
| | | | TCCGGAATTATTGGGCGTAAAGCGCGCGCAGGCGGTTTCTTAAGTCTGATGTGAAAGCCCACGGCTCAA | |
| | | | CCGTGGAAGGTCATTGGAAACTGGGAGACTTGAGTGCAGAAGAGAAGAGCGGAATTCCACGTGTAGCGG | |
| | | | TGAAATGCGTAGAGATGTGGAGGAACACCAGTGGCGAAGGCGGCTCTTTGGTCTGTAACTGACGCTGAG | |
| | | | GCGCGAAAGCGTGGGGAGCGAACAGGATTAGATACCCTGGTAGTCCACGCCGTAAACGATGAGTGCTAA | |
| | | | GTGTTAGAGGGTTTCCGCCCTTTAGTGCTGCAGTTAACGCATTAAGCACTCCGCCTGGGGAGTACGGCC | |
| | | | GCAAGGCTGAAACTCAAAGGAATTGACGGGGGCCCGCACAAGCGGTGGAGCATGTGGTTTAATTCGAAG | |
| | | | CAACGCGAAGAACCTTACCAGGTCTTGACATCCTCTGACCACCCTAGAGATAGGGACTTCCCCTTCGGG | |
| | | | GGACAGAGTGACAGGTGGTGCATGGTTGTCGTCAGCTCGTGTCGTGAGATGTTGGGTTAAGTCCCGCAA | |
| | | | CGAGCGCAACCCTTAACCTTAGTTGCCAGCATTCAGTTGGGCACTCTAAGGTGACTGCCGGTGACAAAC | |
| | | | CGGAGGAAGGTGGGGATGACGTCAAATCATCATGCCCCTTATGACCTGGGCTACACACGTGCTACAATG | |
| | | | GATGGTACAAAGGGTTGCAAGACCGCGAGGTTTAGCTAATCCCATAAAACCATTCTCAGTTCGGATTGC | |
| | | | AGGCTGCAACTCGCCTGCATGAAGCCGGAATCGCTAGTAATCGTGGATCAGCATGCCACGGTGAATACG | |
| | | | TTCCCGGGCCTTGTACACACCGCCCGTCACACCACGAGAGTTTGTAACACCCGAAGTCGGTGGGGTAAC | |
| | | | CCTTACGGGAGCCAGCCGCCGAAGGTGGGACAGATGATTGGGGTGAAGTCGTAACAAGGTAGCGTATCG | |
| | | | GAAGGTGCGGTGGATCA (SEQ ID NO:74) | |
| Halobacillus | 16S | NCIMB | GAGAGTTTGATCCTGGCTCAGGACGAACGCTGGCGGCGTGCCTAATACATGCAAGTCGAGCGCGGGAAG | |
| halophilus | | 2269 | CAAGCGGATCCTTCGGGGGTGAAGCTTGTGGAACGAGCGGCGGACGGGTGAGTAACACGTGGGCAACCT | |
| | | | GCCTGTAAGACCGGAATAACCCCGGGAAACCOGGGCTAATGCCGGATAACACCTACCTTCACCTGAAGG | |
| | | | AAGGTTAAAAGATGGCTTCTCGCTATCACTTACAGATGGGCCCGCGGCGCATTAGCTAGTTGGTGAGGT | |
| | | | AATAGCTCACCAAGGCGACGATGCGTAGCCGACCTGAGAOGGTGATCGGCCACACTGGGACTGAGACAC | |
| | | | GGCCCAGACTCCTACGGGAGGCAGCAGTAGGGAATCTTCCGCAATGGACGAAAGTCTGACGGAGGAACG | |
| | | | CCGCGTGAACGATGAAGGTCTTCGGATCGTAAAGTTCTGTTGTTAGGGAAGAACAAGTACCGTACGAAC | |
| | | | ACAGCGGTACCTTGACGGTACCTAACGAGGAAGCCCCGGCTAACTACGTGCCAGCAGCCGCGGTAATAC | |
| | | | GTAGGGGGCAAGCGTTGTCCGGAATTATTGGGCGTAAAGCGCGCGCAGGCGGTTCTTTAAGTCTGATGT | |
| | | | GAAAGCCCACGGCTCAACCGTGGAGGGTCATTGGAAACTGGGGAACTTGAGGACAGAAGAGGAGAGTGG | |
| | | | AATTCCACGTGTAGCGGTGAAATGCGTAGATATGTGGAGGAACACCAGAGGCGAAGGCGACTCTCTGGT | |
| | | | CTGTTTCTGACGCTGAGGTGCGAAAGCGTGGGTAGCAAACAGGATTAGATACCCTGGTAGTCCACGCCG | |
| | | | TAAACGATGAGTGCTAGGTGTTAGGGGGCTTCCACCCCTTAGTGCTGAAGTTAACGCATTAAGCACTCC | |
| | | | GCCTGGGGAGTACGGCCGCAAGGNTGAAACTCAAAGGAATTGACGGGGGCCCGCACAAGCGGTGGAGCA | |
| | | | TGTGGTTTAATTCGAAGCAACGCGAAGAACCTTACCAGGTCTTGACATCCTTGGAACCACCCTAGAGAT | |
| | | | GGTGTTCCTTCGGGGACCAAGGTGACAGGTGGTGCATGGTTGTCGTCAGCTCGTGTCGTGAGATGTTGG | |
| | | | GTTAAGTCCCGCAACGAGCGCAACCCCTAATCTTAGTTGCCAGCATTCAGTTGGGCACTCTAAGGTGAC | |
| | | | TGCCGGTGACAAACCGGAGGAAGGCGGGGATGACGTCAAATCATCATGCCCCTTATGACCTGGGCTACA | |
| | | | CACGTGCTACAATGGATGGTACAAAGGGCAGCGAAGCCGCGAGGTGTAGCAAATCCCATAAAACCATTC | |
| | | | TCAGTTCGGATTGCAGGCTGCAACTCGCCTGCATGAAGCCGGAATCGGTAGTAATCGCGGATCAGCATG | |
| | | | CCGCGGTGAATACGTTCCCGGGTCTTGTACACACCGCCCGTCACACCACGAGAGTTGGCAACACCC | |
| | | | (SEQ ID NO:75) | |
| Bacillus | 16S | IAM12468 | GACGAACGCTGGCGGCATGCCTAATACATGCAAGTCGAGCGGAATGACGAGAGCTTGCTCTCGATTTTA | |
| psychrophilus | | | GCGGCGGACGGGTGAGTAACACGTGGGCAACCTGCCCTACAGATGGGGATAACTCCGGGAAACCGGGGC | |
| | | | TAATACCGAATAATCAGTTTGTCCGCATGGACAAACTCTGAAAGACGGTTTCGGCTGTCACTGTAGGAT | |
| | | | GGGCCCGCGGCGCATTAGCTAGTTGGTGGGGTAATGGCCTACCAAGGCAACGATGCGTAGCCGACCTGG | |
| | | | AGAGGGTGATCGGCCACACTGGGACTGAGACACGGCCCAGACTCCTACGGGAGGCAGCAGTAGGGAATC | |
| | | | TTCCACAATGGACGAAAGTCTGATGGAGCAATGCCGCGTGAGCGAAGAAGGTTTTCGGATCGTAAAGCT | |
| | | | CTGTTGTAAGGGAAGAACACGTACGGGAGTAACTGCCCGTGCCATGACGGTACCTTATTAGAAAGCCAC | |
| | | | CGGCTAACTACGTGCCAGCAGCCGCGGTAATACGTAGGTGGCAAGCGTTGTCCGGAATTATTGGGCGTA | |
| | | | AAGCGCGCGCAGGCGGTTCTTTAAGTCTGATGTGAAAGCCCACGGCTCACCGTGGAGGGTCATTGGAAA | |
| | | | CTGGAGAACTTGAGTACAGAAGAGGAAAGCGGAATTCCACGTGTAGCGGTGAAATGCGTAGAGATGTGG | |
| | | | AGGAACACCAGTGGCGAAGGCGGCTTTCTGGTCTGTAACTGACGCTGAGGCGCGAAAGCGTGGGGAGCA | |
| | | | AACAGGATTAGATACCCTGGTAGTCCACGCCGTAAACGATGAGTGCTAAGTGTTAGGGGGTTTCCGCCC | |
| | | | CTTAGTGCTGCAGCTAACGCATTAAGCACTCCGCCTGGGGAGTACGGCCGCAAGGCTGAAACTCAAAGG | |
| | | | AATTGACGGGGACCCGCACAAGCGGTGGAGCATGTGGTTTAATTCGAAGCAACGCGAAGAACCTTACCA | |
| | | | GGTCTTGACATCCCACTGACCGGTGTAGAGATACGCCTTTCCCTTCGGGGACAGTGGTGACAGGTGGTG | |
| | | | CATGGTTGTCGTCAGCTCGTGTCGTGAGATGTTGGGTTAAGTCCCGCAACGAGCGCAACCCTTGATCTT | |
| | | | AGTTGCCAGCATTCAGTTGGGCACTCTAAGGTGACTGCCGGTGATAAACCGGAGGAAGGTGGGGATGAC | |
| | | | GTCAAATCATCATGCCCCTTATGACCTGGGCTACACACGTGCTACAATGGATGATACAGAGGGTTGCCA | |
| | | | ACCCGCGAGGGGGAGCCAATCCCATAAAATCGTTCCCAGTTCGGATTGGAGGCTGCAACTCGCCTCCAT | |
| | | | GAAGTTGGAATCGCTAGTAATCGTGGATCAGCATGCCACGGTGAATACGTTCCCGGGTCTTGTACACAC | |
| | | | CGCCCGTCACACCACGAGAGTTTGTAACACCCGAAGTCGGTGGGGTACATCTACGGGAGCCAGCCGCCG | |
| | | | AAGGTGGGACAGATGATTGGGGTGAAGTCGTAACAA (SEQ ID NO:76) | |
| Paenibacillus | 16S | ATCC | TTATTGGAGAGTTTGATCCTGGCTCAGGACGAACGCTGGCGOCGTGCCTAATACATGCAAGTCGAGCGG | |
| macerans | | 8244 | ACCTGATGGAGTGCTTGCACTCCTGATGNNCGGCGGACGGGTGAGTAACACGTAGGCAACCTGCCCGTA | |
| | | | AGACCGGGATAACTACCGGAAACGGTAGCTAATACCGGATAATCAAGTCTTCCGCATAGGAGACTTGGG | |
| | | | AAAGGCGGAGCAATCTNTCACTTACGGANNNNNTNCGGCGCATTAGCTAGTTNGTGGGGTAACGGCTTA | |
| | | | CCAAGGCGACGATGCGTAGCNGACCTGAGAGGGTGAACGGCCACACTGGGACTGAGACACGGCCCAGAC | |
| | | | TCCTACGGGAGGCAGCAGTAGGGAATCTTCCGCAATGGACGAAAGTCTGACGGAGCAACGCCGCGTGAG | |
| | | | TGATGAAGGTTTTCGGATCGTAAAGCTGNNTTGCCAGGGAAGAACGTCTTCTAGAGTAACTGCNANGAG | |
| | | | AGTGACGGTACCTGAGAAGAAAGCCCCGGCTAACTACGTGCCAGCAGCCGCGGTAATACGTAGGGGGCA | |
| | | | AGCGTTGTCCGGAATTATTGGGCGTAAAGCGCGCGCAGGCGGCTGTTTAAGTCTGGTGTATAATCCCGG | |
| | | | GGCTCAACTCCGGGTCGCACTGGAAACTGGACGGCTTGAGTGCAGAAGAGGAGAGTGGAATTCCACGTG | |
| | | | TAGCGGTGAAATGCGTAGAGATGTGGAGGAACACCAGTGGCGAAGGCGACTCTCTNGGCTGTAACTGAC | |
| | | | GCTGAGGCGCGAAAGCGTGGGGAGCAAACAGGATTAGATACCCTGGTAGTCCACGCCGTAAACGATGAG | |
| | | | TGCTAGGTGTTAGGGGTTTCGATACCCTTGGTGCCGAAGTAAACACATTAAGCACTCCGCCTGGGGAGT | |
| | | | ACGGCCGCAAGGCTGAAACTCAAAGGAATTGACGGGGACCCGCACAAGCAGTGGAGTATGTGGTTTAAT | |
| | | | TCGAAGCAACGCGAAGAACCTTACCAGGTCTTGACATCCCTCTGACCGCTGTAGAGATATGGCTTTCTT | |
| | | | CGGGACAGAGGAGACAGGTGGTGCATGGTTGTCGTCAGCTCGTGTCGTGAGATGTTGGGTTAAGTCCCG | |
| | | | CAACGAGCGCAACCCTTGACTTTAGTTGCCAGCAAGTAAAGTTGGGCACTCTAGAGTGACTGCCGGTGA | |
| | | | CAAACCGGAGGAAGGTGGGGATGACGTCAAATCATCATGCCCCTTATGACCTTGNCACACACGTACTAC | |
| | | | AATGGCCGGTACAACGGGAAGCGAAGTAGTGATATGGAGCGAATCCTAGAAAGCCNGTCNCAGTTCGGA | |
| | | | TTGCAGGCTGCAACTCGCCTGCATGAAGTCGGAATTGCTAGTAATCGCGGATCAGCATGCCGCGGTGCG | |
| | | | AATACGTTCCCGGGTNTTGTACACACCGCCCGTCACACCACGAGAGTTTACAACACCCGAAGTCGGTGA | |
| | | | GGTAACCGCAAGGGGCCAGCCGCCGAAGGTGGGGTAGATGATTGGGG (SEQ ID NO:77) | |
| Bacillus | 16S | ATCC | AACGCTGGCGGCGTGCCTAATACATGCAAGTCGAGCGAATTGTTGAGTTTACTCAACAATTAGCGGCGG | |
| psychrosaccha- | | 23296 | ACGGGTGAGTAACACGTGGGCAACCTGCCTATAGACTGGATAACTTCGGGAACCGGAGCTAATACCGAT | |
| rolyticus | | | ATGTTCTTCTCTCGCATGAGAGAAGATGGAAAGACGGTCTCGGCTGTCACTTATAGATGGGCCCGCGGC | |
| | | | GCATTAGCTAGTTGGTGAGGTAATGGCTCACCAAGGCAACGATGCGTAGCCGACCTGAGAGGGTGATCG | |
| | | | GCCACACTGGGACTGAGACACGGCCCAGACTCCTACGGGAGGCAGCAGTAGGGAATCTTCCGCAATGGA | |
| | | | CGAAAGTCTGACGGAGCAACGCCGCGTGAACGATGAAGGCTTTCOGGTCGTAAAGTTCTGTTGTTAGGG | |
| | | | AAGAACAAGTACCAGAGTAACTGCTGGTACCTTGAGGTACCTAACCAGAAAGCCACGGCTAACTACGTG | |
| | | | CCAGCAGCCGCGGTAATACGTAGGTGGCAAGCGTTATCCGGAATTATTGGGCGTAAAGCGCGCGCAGGT | |
| | | | GGTTCCTTAAGTCTGATGTGAAAGCCCCCGGCTCAACCGGGGAGGGTCATTGGAAACTGGGGAACTTGA | |
| | | | GTGTAGAAGAGGAAAGTGGAATTCCACGTGTAGCGGTGAAATGCGTAGAGATGTGGAGGAACACCAGTG | |
| | | | GCGAAGGCGACTTTCTGGTCTATAACTGACACTGAGGCGCGAAAGCGTGGGGAGCAAACAGGATTAGAT | |
| | | | ACCCTGGTAGTCCACGCCGTAAACGATGAGTGCTAAGTGTTAGAGGGTTTCCGCCCTTTAGTGCTGCAG | |
| | | | CTAACGCATTAAGCACTCCGCCTGGGGAGTACGGCCGCAAGGCTGAAACTCAAAGGAATTGACGGGGCC | |
| | | | CGCACAAGCGGTGGAGCATGTGGTTTAATTCGAAGCAACGCGAAGAACCTTACCAGGTCTTGACATCCT | |
| | | | CTGACACTCCTAGAGATAGGACGTTCCCCTTCGGGGGACAGAGTGACAGGTGGTGCATGGTTGTCGTCA | |
| | | | GCTCGTGTCGTGAGATGTTGGGTTAAGTCCCGCAACGAGCGCAACCCTTGATCTTAGTTGCCAGCATTC | |
| | | | AGTTGGGCACTCTAAGGTGACTGCCGGTGATAAACCGGAGGAAGGTGGGGATGACGTCTCATCATGCCC | |
| | | | CTTATGACCTGGGCTACACACGTGCTACAATGGATGGTACAAAGAGCTGCAAACCCGCGAGGGTAAGCG | |
| | | | ATCTCATAAAGCCATTCTCAGTTCGGATTGCAGGCTGCAACTCGCCTGCATGAAGCCGGAATCGCTAGT | |
| | | | AATCGCGGATCAGCATGCCGCGGTGAATACGTTCCCGGGCCTTGTACACACCGCCCGTCACACCACGAG | |
| | | | AGTTTGTAACACCCGAGTCGGTGAGGTAACCGCAAGGAGCCAGCCCGCCTAAGGTGGGACAGATGATTG | |
| | | | GGGTGAAGTCGTAACAAGGTAGCCGTATCGGAAGGTGCGGCTGGATCA (SEQ ID NO:78) | |
| Bacillus | 16S | ATCC | AACGCTGGCGGCGTGCCTAATACATGCAAGTCGAGCGAATGGATTAAGAGCTTGCTCTTATGAAGTTAG | |
| mycoides | | 6462 | CGGCGGACGGGTGAGTAACACGTGGGTAACCTACCCATAAGACTGGGATAACTCCGGGAAACCGGGGCT | |
| | | | AATACCGGATAATATTTTGAACTGCATAGTTCGAAATTGAAAGGCGGCTTCGGCTGTCACTTATGGATG | |
| | | | TGGACCCGCGTCGCATTAGCTAGTTGGTGAGGTAACGGCTCACCAAGGCGACGATGCGTAGCCGACCTG | |
| | | | AGAGGGTGATCGGCCACACTGGGACTGAGAACGGCCCAGAGTCCTACGGGAGGCAGCAGTAGGGAATCT | |
| | | | TCCGCAATGGACGAAAGTCTGACGGAGCAACGCCGCGTGAGTGATGAAGGCTTTCGGGTCGTAAAACTC | |
| | | | TGTTGTTAGGGAAGAACAAGTGCTAGTTGAATAAGCTGGCACCTTGAGCGCGCGCAGGTGGTTTCTTAA | |
| | | | GTCTGATGTGAAAGCCCACGGCTCAACCGTGGAGGGTCATTGGAAACTGGGAGACTTGAGTGCAGAAGA | |
| | | | GGAAAGTGGAATTCCATGTGTAGCGGTGAATGCGTAGAGATATGGAGGAACACCAGTGGCGAAGGCGAC | |
| | | | TTTCTGGTCTGTAACTGACACTGAGGCGCGAAAGCGTGGGGAGCAAACAGGATTAGATACCCTGGTAGT | |
| | | | CCACGCCGTAAACGATGAGTGCTGAAGTGTTAGAGGGTTTCCGCCCTTTAGTGCTGAAGTTAACGCATT | |
| | | | AAGCACTCCGCCTGGGGAGTACGGCCGCAAGGCTGAAACTCAAGGAATTGACGGGGGCCCGCACAAGCG | |
| | | | GTGGAGCATGTGGTTTAATTCGAAGCAACGCGAAGAACCTTACCAGGTCTTGACATCCTCTGAAAACTC | |
| | | | TAGAGATAGAGCTTCTCCTTCGGAGCAGAGTGAAGGTGGTGCATGGTTGTCGTCCTCGTGTCGTGAGAT | |
| | | | GTTGGGTTAAGTCCCGCAACGAGCGCAACCCTTGATTAGTTGCCATCATTAAGTTGGGCACTCTAAGGT | |
| | | | GACTGCCGGTGACAAACCGGAGGAAGGTGGGGATGACGTCAAATCATCATGCCCCTTATGACCTGGGCT | |
| | | | ACACACGTGCTACAATGGACGGTACAAAGAGCGCAAGACCGCGAGGTGGAGCTAATCTCATAAAACCGT | |
| | | | CTCAGTTCGGATTGTAGGCTGCAACTCGCCTACATGAAGCTGGAATCGCTAGTAATCGCGGATCAGCAT | |
| | | | GCCGCGGTGAATACGTTCCCGGGCCTTGTACACACCGCCCGTCACACCACGAGAGTTTGTAACACCCGA | |
| | | | AGTCGGTGGGGTAACCTTTATGGAGCCAGCCGCCTAAGGTGGGACAGATGATTGGGGTGAATGCGTAAC | |
| | | | AAGGTAGCCGTATCGGAAGGTGCGGCTGGATCA (SEQ ID NO:79) | |
| Bacillus | 16S | DSM 485 | GACGAACGCTGGCGGCGTGCCTAATACATGCAAGTCGAGCGGACCAAAGGGAGCTTGCTCCCAGAGGTT | |
| alcalophilus | | | AGCGGCGGACGGGTGAGTAACACGTGGNCAACCTGCCCTGTAGACTGGGATAACATCGAGAAATCGGTG | |
| | | | CTAATACCGGATAATCAAAGGAATCACATGGTTCTTTTGTAAAAGATGGCTCCGGCTATCACTANGGGA | |
| | | | TGGCCCGCGCGCATTAGCTAGTTGGTAAGGTAATGGCTTACCAAGGCGACGATGCGTAGCCGACCTGAG | |
| | | | AGGGTGATCGGCCACACTGGGACTGAGACACGGCCCAGACTCCTACGGGAGGCAGCAGTAGGGAATCTT | |
| | | | CCGCAATGGACGAAAGTCTGACGGAGCAACGCCGCGTGAGTGATGAAGGTTTTCGGATCGTAAAGCTCT | |
| | | | GTTGTTAGGGAAGAACAAGTGCCGNTCGAATAGGTCGGCACCTTGACGGTACCTAACCAGAAAGCCACG | |
| | | | GCTAACTACGTGCCAGCAGCCGCGGTAATACGTAGGTGGCAAGCGTTGTCCGGAATTATTGGGCGTAAA | |
| | | | AAGCGCGCGCAGGCGGTCTTTTAAGTCTGATGTGAAATATCGGGGCTCAACCCCGAGGGGTCATTGGAA | |
| | | | ACTGGGAGACTTGAGTACAGAAGAGGAGAGTGGAATTCCACGTGTAGCGGTGAAATGCGTAGATATGTG | |
| | | | GAGGAACACCAGTGGCGAAGGCGACTCTCTGGTCTGTAACTGACGCTGAGGCGCGAAAGCGTGGGGAGC | |
| | | | AAACAGGATTAGATACCCTGGTAGTCCACGCCGTAAACGATGAGTGCTAGGTGTTAGGGGTTTCGATGC | |
| | | | CCTTAGTGCCGAAGTTAACACATTAAGCACTCCGCCTGGGGAGTACGGCCGCAAGGCTGAAACTCAAAG | |
| | | | GAATTGACGGGGGCCCGCACAAGCAGTGGAGCATGTGGTTTAATTCGAAGCAACGCGAAGAACCTTACC | |
| | | | AGGTCTTGACATCCTTTGACCACTCTAGAGATAGAGCTTTCCCCTTCGGGGGACAAAGTGACAGGTGGT | |
| | | | GCATGGTTGTCGTCAGCTCGTGTCGTGAGATGTTGGGTTAAGTCCCGCAACGAGCGCAACCCTTGATCT | |
| | | | TAGTTGCCAGCATTTAGTTGGGCACTCTAAGGTGACTGCCGGTGACAAACCGGAGGAAGGTGGGGATGA | |
| | | | CGTCAAATCATCATGCCCCTTATGACCTGGGCTACACACGTGCTACATGGATGGTACAAAGGGAGCGAC | |
| | | | CGCGAGGTCGAGCCAATCCCATAAAGCCATTCTCAGTTCGGATTGTAGGCTGCAACTCGCCTACATGAA | |
| | | | GCCGGAATTGCTAGTAATCGCGGATCAGCATGCCGCGGTGAATACGTTCCCGGGCCTTGTACACACCGC | |
| | | | CCGTCACACCACGAGAGTTTGTAACACCCGAAGTCGGTGGGGTAACGTTTTGGAGCCAGCCGCCTAAGG | |
| | | | TGGGACAGATGATTGGGGTGAAGTCGTAACAAGGTAGCCGTATCGGAAGGTG (SEQ ID NO:80) | |
| Aneuriniba- | 16S | ATCC | GAGAGTTTGATCCTGGCTCAGGNCGANCGCTGGCGGTGTGCCTAATACATGCAAGNCGAGCGGACCAAG | |
| cillus | | 12856 | GAAGAGCTTGCTCTTCGGCGGTTAGCGGCGGACGGGTGAGTAACACGTAGGCAGCCTGCCTGTACGACT | |
| aneurinilyti- | | | GGGATAACTCCGTGAAACCGGAGCTAATACCAGATACGTTTTTCAGACCGCATGGTCTGAAAGAGAAAG | |
| cus | | | ACCTCTGGTCACGTACAGATGGGCCTGCGGCGCATTAGCTAGTTGGTGGGGTAACGGTCTACCAAGGCG | |
| | | | ACGATGCGTAGCCGACCTGAGAGGGTGATCGGCCACACTGGGACTGAGACACGGCCCAGACTCCTACGG | |
| | | | GAGGCAGCAGTAGGGAATCTTCCGCAATGGACGAAAGTCTGACGGAGCAACGCCGCGTGAACGATGAAG | |
| | | | GTTTTCGGATCGTAAAGTTCTGTTGTTAGGGAAGAACCGCCGGGATGACCTCCCGGTCTGACGGTACCT | |
| | | | AACGAGAAAGCCCCGGCTAACTACGTGCCAGCAGCCGCGGTAATACGTAGGGGGCAAGCGTTGTCCGGA | |
| | | | ATTGGGCGTAAAGCGCGCGCAGGCGGCTTCTTAAGTCAGGTGTGAAAGCCCACGGNTCAACCGTGGAGG | |
| | | | GCCACTTGAAACTGGGAGGCTTGAGTGCAGGAGAGGAGAGCGGAATTCCACGTGTAGCGGTGAAATGCG | |
| | | | TAGAGATGTGGAGGAACAACCGTGGCGAAGGCGGCTCTCTGGCCTGTAACTGACGCTGGGGCGCGAAAG | |
| | | | CGTGGGGAGCGAACAGGATTAGATACCCTGGTAGTCCACGCCGAAAACGTTGAGTGTTAGGTGTTGGGG | |
| | | | ACTCCAATCCTCAGTGCCGCAGCTAACGCAATAAGCACTCCGCCTGGGGAGTACGGCCGCAAGGCTGAA | |
| | | | ACTCAAAGGAATTGACGGGGACCCGCACAAGCGGTGGAGCATGTGGTTTAATTCGAAGCAACGCGAAGA | |
| | | | ACCTTGCCAGGGCTTGACATCCCGCTGTCCCTCCTAGAGATAGGAGNTCTCTTCGGAGCAGCGGTGACA | |
| | | | GGTGGTGCATGGTTGTCGNCAGCTCGTGTCGTGAGATGTTGGGTTAAGTCCCGCAACGAGCGCAACCCT | |
| | | | TGTCCTTAGTTGNCAGCATTCAGTTGGGCACTCTAGGGAGACTGCCGTCGACAAGACGGAGGAAGGTGG | |
| | | | GGATGACGTCAAATCATCATGCCCCTTATGTCCTGGGCTACACACGTGCTACAATGGATGGAACAACGG | |
| | | | GCAGCCAACTCGCGAGAGTGCGCCAATCCCTTAAAACCATTCTCAGTTCGGATTGCAGGCTGCAACCTC | |
| | | | GCCTGCATGAAGCCGGAATCGCTAGTAATCGCGGATCAGCATGCCGCGGTGAATACGTTCCCGGGTCTT | |
| | | | GTACACACCGCCCGTCACACCACGAGAGTTTGCAACACCC (SEQ ID NO:81) | |
| Amphibacillus | 16S | DSM 6626 | ATCCTGGCTCAGGATGAACGCTGGCGGCGTGCCTAATACATGCAAGTCGAGCGCGTCNNATTAAAACAGA | |
| xylanus | | | TCTCTTCGGAGTGACGTTTAATGGATCGAGCGGCGGATGGGTGAGTAACACGTGGCCAACCTGCCTATAA | |
| | | | GACTGGGATAACTTACGGAAACGTGAGCTAATACCGGATAAAACCTTTTGTCTCCTGACAAGAGGATAAA | |
| | | | AGATGGCGCAAGCTATCACTTATAGATGGGCCCGCGGCGCATTAGCTAGTTGGTGAGATAAAAGCTCACC | |
| | | | AAGCCACGATGCGTAGCCGACCTGAGAGGGTGATTGGCCACACTGGGACTGAGATACGGCCCGATCCTAC | |
| | | | GGGAGGCAGCAGTAGGGAATCTTCCGCAATGGACGAAAGTCTGACGGAGCAACGCCGCGTGAACGAAGAA | |
| | | | GGTCTTCGGATCGTAAAGTTCTGTTGTTAGGGAAGAACACGTACCATTCGAATAGGGTGGTACCTTGACG | |
| | | | GTACCTAACGAGAAAGCCCCGGCTAACTACGTGCCAGCAGCCGCGGTAATACGTAGGGGGCAAGCGTTGT | |
| | | | CCGGAATTATTGGGCGTAAAGCGCGCGCAGGCGGTTCTTTAAGTCTGATGTGAAATCTTGCGGCTCAACC | |
| | | | GCAAGCGGTCATTGGAAACTGGAGAACTTGAGGACAGAAGAGGAGAGTGGAATTCCACGTGTAGCGGTGA | |
| | | | ATGCGTAGAGATGTGGAGGAACACCAGTGGCGAAGGCGACTCTCTGGTCTGTAACTGACGCTGAGGCGCG | |
| | | | AAAGCGTGGGTAGCGAACAGGATTAGATACCCTGGTAGTCCACGCCGTAAACGATGAGTGCTAGGTGTTA | |
| | | | GGGGGTTTCCCCCCCTTAGTGCTGGCGTTAACGCATTAAGCACTCCNCCTGGGGAGTACGGCCGCAAGGC | |
| | | | TGAAACTCAAAAGAATTGACGGGGACCCGCACAAGCGGTGGAGCATGTGGTTTAATTCGAAGCAACGCGA | |
| | | | AGAACCTTACCAGGTCTTGACATCCCGCTGACCGCTATGGAGACATAGCTTTCCCTTCGGGGACAGCGGT | |
| | | | GACAGGTGGTGCATGGTTGTCGTTGCTCGTGTCGTGAGATGTTGGGTTAAGTCCCGCAACGAGCGCAACC | |
| | | | CTTGAACTTAGTTGCCAGCATTCAGTTGGGCACTCTAAGTTGACTGCCGGTGACAAACCGGAGGAAGGTT | |
| | | | GGGATGACGTCAAATCATCATGCCCCTTATGACCTGGGCTACACACGTGCTACAATGGNTTGGTAGTTCG | |
| | | | GATTGTCGGTTGAACTCGCCTACATGAAGCCGGAATCGCTAGTAATCGCGGATCAGAATGCCGCGGTGAA | |
| | | | TACGTTCCCGGGTCTTGTACACACCGTCCGTCACACCACGAGAGTTAGCAACACCCGAAGTCGGTGAGGT | |
| | | | AACGCTTTTAGNGAGCCAGCCGCCGAAGGTGGGGCCAATGATTGGGGTGAAGTCGTAACAAGGTAGCCGT | |
| | | | ATCGGAAGGTGCGGNTGGATCACCTCCTT (SEQ ID NO:82) | |
| Bacillus | 16S | IAM | GACGAACGCTGGCGGCGTGCCTAATACATGCAAGTCGAGCGCGGGAACNAAGCAGATCTCCTTCGGGGGT | |
| pantothenticus | | 11061 | GACGCTTGTCCAACGGACGGGTGAGTAACACGTGGGCAACCTACCTGTAAGACTGGGATAACTCCGGGAA | |
| | | | ACCGGGGCTAATACCGGATGATACATATCGTCCATACGAGATGTTGAAAAGGCGGCATATGCTGTCACTT | |
| | | | ACAGATGGGCCCGCGGCGCATTAGCTAGTTGGTGAGATAAAAGCTCACCAAGGCGACGATGCGTAGCCGA | |
| | | | CCTGAGAGGGTGATCGGCCACACTGGGACTGAGACACGGCCCAGACTCCTACGGGAGGCAGCAGTAGGGA | |
| | | | ATCTTCCGCAATGGACGAAAGTCTGACGGAGCAACGCCGCGTGAGTGATGAAGGTTTTCGGATCGTAAAA | |
| | | | CTCTGTTGTTAGGGAAGAACAAGTGCCATTCGAATAGGTTGGCACCTTGACGGTACCTAACCAGAAAGCC | |
| | | | CCGGCTAACTACGTGCCAGCAGCCGCGGTAATACGTAGGGGGCAAGCGTTGTCCGGAATTATTGGGCGTA | |
| | | | AAGCGCGCGCAGGCGGTCCTTTAAGTCTGATGTGAAAGCCCACGGCTTAACCGTGGAGGGCCATTGGAAA | |
| | | | CTGGGGGACTTGAGTACAGAAGAGGAGAGTGGAATTCCACGTGTAGCGGTGAAATGCGTAGAGATGTGGA | |
| | | | GGAACACCAGTGGCGAAGGCGACTCTCTGGTCTGTAACTGACGCTGAGGTGCGAAAGCGTGGGTAGCGAA | |
| | | | CAGGATTAGATACCCTGGTAGTCCACGCCGTAAACGATGAGTGCTAGGTGTTAGGGGGTTTCCGCCCCTT | |
| | | | AGTGCTGAAGTTAACGCATTAAGCACTCCGCCTGGGGAGTACGGTCGCAAGACTGAAACTCAAAGGAATT | |
| | | | GACGGGGACCCGCACAAGCGGTGGAGCATGTGGTTTAATTCGAAGCAACGCGAAGAACCTTACCAGGTCT | |
| | | | TGACATCCTCTGACGCCCCTAGAGATAGGGAGTGATCTTAGTTGCCAGCATTTAGTTGGGCACTCTAAGG | |
| | | | TGACTGCCGGTGACAAACCGGAGGAAGGTGGGGATGACGTCAAATCATCATGCCCCTTATGACCTGGGCT | |
| | | | ACACACGTGCTACAATGGATGGAACAAAGGGCAGCGAAGCCGCGAGGCCAAGCAAATCCCATAAAACCAT | |
| | | | TCTCAGTTCGGATTGCAGGCTGCAACTCGCCTGCATGAAGCCGGAATCGCTAGTAATCGCGGATCAGCAT | |
| | | | GCCGCGGTGAATACGTTCCCGGGTCTTGTACACACCGCCCGTCACACCACGAGAGTTGGTAACACCCGAA | |
| | | | GTCGGTGAGGTAACCTTTTGGAGCCAGCCGCCGAAGGTGGGACTAATGATTGGGGTGAAGTCGTAACAA | |
| | | | (SEQ ID NO:83) | |
| Paenibacillus | 16S | ATCC | TGCCTAATACATGCAAGTCGAGCGGACTCAACTGTTTCCTTCGGGAAACCGTTAGGTTAGCGGCGGACGG | |
| popilliae | | 14706 | GTGAGTAATACGTAGGTAACCTGCCCTTAAGACYGGGATAACTCACGGAAACGTGGGCTAAWACCGGATA | |
| | | | GGCGATTTGCTCGCATGAGGGAATCGGGAAAGGCGGAGCAATCTGCCACTTATGGATGGACCTACGGCGC | |
| | | | ATTAGCTAGTTGGTGRGGTAACGGCTCACCAAGGCGACGATGCGTAGCCGACCTGAGAGGGTGATCGGCC | |
| | | | ACACTGGGACTGAGACACGGCCCAGACTCCTACGGGAGGCAGCAGTAGGGAATCTTCCGCAATGGACGCA | |
| | | | AGTCTGACGGAGCAACGCCGCGTGAGTGATGAACGTTTTCGGATCGTAAAGCTCTGTTGCCAGGGAAGAA | |
| | | | CGCTATGGAGAGTAACTGTTCCATAGGTGACGATACCTGAGAAGAAAGCCCCGGCTAACTACGTGCCAGC | |
| | | | AGCCGCGGTAATACGTAGCGGGGCAAGCGTTGTCCGGAATTATTGGGCGTAAAGCGCGCGCAGGCGGTCA | |
| | | | TGTAAGTCTGGTGTTTAAACCCGGGGCTCAACTCCGGGTCGCATCGGAAACTGTGTGACTTGAGTGCAGA | |
| | | | AGAGGAAAGTGGAATTCCACGTGTAGCGGTGATGCGTAGAGATGTGGAGGAACACCAGTGGCGAAGGCGA | |
| | | | CTTTCTGGGCTGTAACTGACGCTGAGGCGCGAAAGCGTGGGGAGCAACAGGATTAGATACCCTGGTAGTC | |
| | | | CACGCCGTAAACGATGAATGCTAGGTGTTAGGGGTTTCGATACCCTTGGTGCCGAAGTTAACACATTAAG | |
| | | | CATTCCGCCTGGGGAGTACGGTCGCAAGACTGAAACTCAAAGGAATTGACGGGGACCCGCACAAGCAGTG | |
| | | | GAGTATGTGGTTTAATTCGAGCAACGCGAAGAACCTTACCAGGTCTTGACATCCCTCTGACCGCGCTAGA | |
| | | | GATAGGGCTTCCCTTCGGGGCAGAGGAGACAGGTGGTGCATGGTTGTCGTCAGCTCGTOTCGTGAGATGT | |
| | | | TGGGTTAAGTCCCGCAACGAGCGCAACCCTTAACTTTAGTTGCCAGCATTGAGTTGGGCACTCTAGAGTG | |
| | | | ACTGCCGGTGAAACCGGAGGAAGGTGGGGATGACGTCAAATCATCATGCCCCTTATGACCTGGGCTACAC | |
| | | | ACGTACTACAATGGCTGGTACAACGGGAAGCGAAGCCGCGAGGTGGAGCGAATCCTAAAAAGCCAGTCTC | |
| | | | AGTTCGGATTGCAGGCTGCAACTCGCCTGCATGAAGTCGGAATTGCTAGTAATCGCGGATCAGCATGCCG | |
| | | | CGGTGAATACGTTCCCGGGTCTTGTACACACCGCCCGTCACACCACGAGAGTTTACAACACCCGAAGTCG | |
| | | | GTGGGGTAACCGCAAGGAGCCAGCCGCCGAAGGTGGGGTAGATGATTGGGGTGAAGTCGTAACAA | |
| | | | (SEQ ID NO:84) | |
| B. cereus | cpn60 | | GCAACTGTATTAGCGCAAGCTATGATTCGTGAAGGTCTTAAAAACGTAACAGCTGGTGCGAACCCAATGG | |
| | | | GGCTTCGTAAAGGTATCGAAAAAGCTGTTACTGCTGCAATTGAAGAATTAAAAACGATTTCTAAACCAAT | |
| | | | CGAAGGCAAATCTTCTATCGCACAAGTAGCTGCTATTTCTGCAGCTGACGAAGAGTAGGTCAATTAATCG | |
| | | | CTGAAGCAATGGAGCGCGTTGGTAACGACGGCGTTATTACTTTAGAAGAGTCTAAAGGATTCACAACAGA | |
| | | | ATTAGACGTAGTAGAAGGTATGCAATTTGATCGTGGATATGCATCTCCTTACATGATTACTGATTCTGAC | |
| | | | AAAATGGAAGCAGTTCTTGATAACCCATACATCTTAATTACTGACAAAAAGATTTCTAACATTCAAGAAA | |
| | | | TCTTACCAGTATTAGAGCAAGTGGTACAACAAGGTAAACCACTTCTTATCATTGCTGAAGATGTAAAAGG | |
| | | | CGAAGCTTTAGCTACATTAGTAGTGAACAAACTTCGTGGTACATTCAACGTAGTAGCTGTT | |
| | | | (SEQ ID NO:85) | |
| .thuringien- | Cpn60 | | GCAACTGTATTAGCGCAAGCTATGATTCGTGAAGGTCTTAAAAACGTAACAGCTGGTGCGAACCCAATGG | |
| sis var. | | | GTCTTCGTAAAGGTATCGAAAAAGCTGTTACTGCTGCAATTGAAGAATTAAAAACGATTTCTAAACCAAT | |
| kurstaki | | | CGAAGGTAAATCTTCTATCGCACAAGTAGCTGCTATTTCTGCTGCTGACGAAGAAGTAGGTCAATTAATC | |
| HD1 (=B51 B. | | | CTGAAGCAATGGAGCGCGTTGGTAACGACGGCGTTATTACTTTAGAAGAATCTAAAGGCTTCACAACAGA | |
| anthracis) | | | ATTAGACGTAGTAGAAGGTATGCAATTTGATCGTGGATATGCATCTCCTTACATGATTACTGATTCTGAC | |
| | | | AAAATGGAAGCAGTTCTTGATAACCCATACATCTTAATCACTGACATAAGATTTCTAACATTCAAGAAAT | |
| | | | CTTACCAGTATTAGAGCAAGTGGTACAACAAGGTAAACCACTTCTTATCATTGCTGAAGATGTAGAGGCG | |
| | | | AGCGTTAGCTACATTAGTAGTGAACAAACTTCGTGGTACATTCAATGTAGTAGCTGTT | |
| | | | (SEQ ID NO:86) | |
| B. subtilis | cpn60 | | GCAACAGTTCTTGCGCAAGCAATGATCCGTGAAGGCCTTAAAAACGTAACAGCAGGCGCTAACCCTGTAG | |
| 168 | | | GCGTGCGTAAAGGGATGGAACAAGCTGTAGCGGTTGCGATCGACTTAGAAATTTCTAAGCCAATCGAAGG | |
| | | | CAAAGAGTCTATCGCTCAGGTTGCTGCGATCTCTGCTGCTGATGAGGAAGTCGGAAGCCTTATCGCTGAA | |
| | | | GCAATGGAGCGCGTAGGAAACGACGGCGTTATCACAATCGAAGAGTCTAAAGGCTTCACAACTGAGCTTG | |
| | | | ACTGAGCTTGAAGTTGTTGAAGGTATGCAATTCGACCGCGGATATGCGTCTCCTTACATGGTAACTGACT | |
| | | | CTGATAAGATGGAAGCGGTTCTTGACAATCCTTACATCTTAATCACAGACAAAAAAATCACAAACATTCA | |
| | | | AGAAATCCTTCCTGTGCTTGAGCAGGTTGTTCAGCAAGGCAAACCATTGCTTCTGATCGCTGAGGATGTT | |
| | | | GAAGGCGAAGCACTTGCTACACTTGTTGTGAACAAACTTCGCGGCACATTCAACGCAGTGGCTGTT | |
| | | | (SEQ ID NO:87) | |
| B. subtilis | cpn60 | | GCGACAGTTCTTGCGCAAGCAATGATCCGTGAAGGCCTTAAAAACGTAACAGCAGGCGCTAATCCTGTAG | |
| W235R | | | GCGTTCGTAAAGGTATGGAAAAAGCTGTAGCGGTTGCGATCGAAAACTTAAAAGAAATTTCTAAGCCAAT | |
| | | | CGAAGGCAAGGAGTCTATCGCTCAGGTTGCTGCGATCTCTGCTGCTGAGGAGGAAGTCGGAAGCCTTATC | |
| | | | GCTGAAGCAATGGAGCGCGTAGGCAACGACGGCGTTATCACAATCGAAGAGTCTAAAGGCTTCACAACTG | |
| | | | AGCTTGAAGTTGTTGAAGGTATGCAATTCGACCGCGGATATGCGTCTCCTTACATGGTAACTGACTCTGA | |
| | | | TAAGATGGAAGCGGTTCTTGACAATCCTTACATCTTAATCACAGACAAAAAAATCACAAACATTCAAGAA | |
| | | | ATCCTTCCTGTACTTGAGCAGGTTGTTCAGCAAGGCAAACCATTGCTTCTAATCGCTGAGGATGTTGAAG | |
| | | | GCGAAGCACTTGCAACACTTGTTGTGAACAAACTTCGCGGTACATTCAACGCAGTTGCTGTT | |
| | | | (SEQ ID NO:88) | |
| B. licheni- | cpn60 | ATCC14580 | GCGACAGTTCTAGCTCAGGCGATGATTCGCGAAGGTCTTAAAAACGTAACTGCCGGCGCTAACCCTGTAG | |
| formis | | | GCGTGCGTATCGAGCAGGCTGTGGCTGTAGCTGTTGAAAGCCTGAAAGAAATCTCTAAACCAATTGAAGG | |
| | | | CAAAGAATCAATCGCACAAGTTGCTTCAATCTCCGCTGCAGACGAAGAAGTCGGAAGCCTGATCGCTGAA | |
| | | | GCAATGGAGCGCGTCGGCAACGACGGTGTTATCACGATCGAAGAATCCAAAGGATTCACAACAGAGCTTG | |
| | | | AAGTGGTTGAAGGTATGCAGTTCGACCGCGATATGCGTCTCCTTACATGGTGACGGATTCCGATAAGATG | |
| | | | GAAGCGGTTCTTGAGAATCCGTACATCTTAGTAACAGACAAAAAAATCACAAACATTCAAGAAATCCTGC | |
| | | | CGGTGCTTGAGCAAGTCGTGCAACAAGGCAAACCGTTGCTTCTGATTGCTGAAGACGTTGAAGGTGAAGC | |
| | | | TCTTGCAACATTGGTTGTCCAAGCTTCGCGGAACATTCAACGCAGTGGCTGTT (SEQ ID NO:89) | |
| B. pumilus | cpn60 | | GCGACTGTACTTGCGCAGGCTATGATCCGCGAAGGCCTTAAAAACGTAACTGCGGGGGCTAACCCTGTCG | |
| B205-L M&G | | | GCGTGCGTAAAGGTATGGAACA AGCCGTGACTGTAGCAATCGAAAACTTAAAAGAAATTTCTAAGCCGA | |
| | | | TCGAAGGCGAGTCTATCGCTCAGGTTGCTGCGATCTCTGCTGCTGATGAGGAAGTCGGAAGCCTTATCGC | |
| | | | TGAAGCAATGGAGCGCGTAGTAAACGACGGCGTCATCACAATCGAAGAGTCTAAAGGTTTCACA | |
| | | | ACTGAGCTTGAAGTTGTTGAAGGTATGCAATTCGACCGCGATATGCGTCTCCTTACATGGTGACTGACTC | |
| | | | TGATAAGATGGAAGCGGTTCTTGACAATCCTTACATCTTAATCACAGACAAAAAAATCACAAACATTCAA | |
| | | | GAAATCCTTCCTGTGCTTGAGCAAGTTGTACAGCAAGGCAAACCATTGCTTCTGATCGCTGAAGATGTTG | |
| | | | AAGGGGAAGCTCTTGCTACACTCGTTGTCAACAAACTTCGCGGCACATTCAACGCTGTTGCCGTT | |
| | | | (SEQ ID NO:90) | |
| B. pumilus | cpn60 | ATCC7061 | GCAACAGTTCTAGCTCAAGCGATGATCCGTGAAGGTCTTAAAAACGTAACAGCTGGTGCAAACCCTGTTG | |
| | | | GCGTTCGTAAAGGGTATCGAAGAAGCCGTGACTGTAGCAATCGAAAACTTAAAAGAAATTTCTAAGCCGA | |
| | | | TCGAAGGCGTTCGTAAAGGGATCGAAGAAGTTGGAAGCCTGATCGCTGAAGCAATGGAGCGTGTAGGTAA | |
| | | | CGACGGCGTGATCACAATCGAAGAATCTAAAGGGTTCACAACTGAGCTTGAAGTGGTTGAAGGATGCAGT | |
| | | | TTGACCGAGGATATGCTTCACCATACATGGTGACGCTGATAAGATGGAAGCGGTTCTTGAAAATCCTTAC | |
| | | | ATCTTAATCACTGATAAAAAAATCACAAACATTCAAGAAATCCTTCCTGTACTTGAGCAAGTTGTACAAC | |
| | | | AAGGAAAACCATTATTGCTCATTGCTGAAGATGTAGAAGGCGAAGCACTTGCAACACTTGTTGTGAACAA | |
| | | | ACTTCGTGGAACATTCAACGCAGTGGCAGTA (SEQ ID NO: 91) | |
| B. amylolique- | cpn60 | | GCGACTGTGCTTGCACAGGCTATGATCCGCGAAGGCCTTAAACGTAACTGCGGGAGCTAATCCTGTCGGC | |
| faciens H | | | GTGCGTAAAGGTATGGAACAAGCCGTAACCGTGGCAATCGAAAACTTAAAAGAAATTTCTAAGCCGATCG | |
| | | | AAGGCAAAGAGTCTATCGCTCAGGTTGCTGCAATCTCTGCTGCTGATGAGGAAGTCGGAAGCCTTATCGC | |
| | | | TGAAGCAATGGAGCGCGTAGGAAACGACGGCGTTATCACAATCGAAGAGTCTAAAGGCTTCACAACTGAG | |
| | | | CTTGAAGTGGTTGAAGGTATGCAATTCGACCGCGGATATGCGTCTCCTTACATGGTTGACTGACTCTGAT | |
| | | | AAGATGGAAGCGGTTCTTGATAATCCTTACATCTTAATCACAGACAAAAAAATCACAAACATTCAAGAAA | |
| | | | TCCTTCCTGTGCTTGAGCAAGTTGTACAGCAAGGCAAACCATTGCTTCTGATCGCTGAAGATGTTGAAGG | |
| | | | TGAAGCTCTTGCTACACTCGTTGTCAACAAACTTCGCGGCACATTCAACGCTGTTGCCGTT | |
| | | | (SEQ ID NO:92) | |
| B. amylolique- | cpn60 | | GCAACTGTATTAGCACAAGCTATGATTCGTGAAGGTCTTAAAAACGTAACAGCTGGTGCGAACCCAATGG | |
| faciens N | | | GTCTTCGTAAAGGTATCGAAAAAGCTGTAGTTGCTGCAGTAGAAGAATTAAAAACGATTTCTAAACCAAT | |
| | | | CGAAGGTAAATCTTCAATCGCACAAGTAGCTGCTATTTCTGCGGCTGACGAAGAAGTAGGTCTTTAATC | |
| | | | GCTGAAGCAATGGAGCGCGTTGGTAACGACGGCGTTATTACTTTAGAAGAATCTAAAGGATTCACAACAG | |
| | | | AATTAGATGTAGTAGAAGGTATGCAATTTGATCGTGGATATGCATCTCCTTACATGATTACTGATTCTGA | |
| | | | CAAAATGGAAGCAGTTCTTGATAACCCATACATCTTAATCACTGACAAAAAGATTTCTAACATTCAAGAA | |
| | | | ATCTTACCAGTATTAGAGCAAGTGGTACAACAAGGTAAACCGCTTCTTATCATTGCTGAAGATGTAGAAG | |
| | | | GCGAAGCATTAGCTACATTAGTAGTGAACAAACTTCGTGGTACATTCAATGTAGTAGCTGTT | |
| | | | (SEQ ID NO:93) | |
| B. globigii | cpn60 | | GCTACAGTTCTTGTTCAGGCTATGATTCGTGAAGGTCTTAAAAACGTAACGGCAGGCGCTAACCCTGTAG | |
| SB512 | | | GCGTTCGTAAAGGTATGGAACAAGCTGTAACAGTTGCGATTCAAACCTTCAAGAAATCTCTAAACCGATC | |
| | | | GAAGGAAAAGAGTCTATCGCTCAGGTTGCTGCGATTTCTGCTGCTGATGAAAAAGTCGGAAGCCTGATTG | |
| | | | CTGAAGCGATGGAGCGCGTTGGAAACGACGGCGTTATCACGATCGAAGAATCTAAAGGTTTCACAACTGA | |
| | | | GCTTGAAGTTGTTGAAGGTATGCAGTTCGACCGCGGATATGCATCTCCTTACATGGTAACTGATTCTGAT | |
| | | | AAGATGGAAGCGGTTCTTGAAAATCCTTACATCTTAATCACAGACAAAAAAATTACAAATATTCAAGAAA | |
| | | | TCCTTCCTGTGCTTGAGCAGGTTGTTCAGCAAGGCAAACCATTGCTTCTGATTGCTGAGGATGTTGAAGG | |
| | | | TGAAGCTCTTGCAACACTTGTTGTGAACAAACTTCGCGGCACATTCAACGCAGTTGCCGTT | |
| | | | (SEQ ID NO:94) | |
| G. stearother- | cpn60 | | GCAACAGTTTTAGCGCAAGCAATGATCCGCGAAGGATTGAAAAACGTTACAGCTGGCGCTAACCCAATGG | |
| mophilus | | | GCATCCGTAAAGGTATTGAAAAAGCGGTCGCTGTGGCAGTAGAAGAATTAAAAGCAATCTCCAAACCAAT | |
| BGSC strain | | | TCAAGGTAAAGAATCGATTGCTCAAGTTGCAGCGATCTCTGCGGCTGACGAAGAAGTTGGTCAATTAATC | |
| 9A2 | | | GCAGAAGCAATGGAACGCGTTGGCAACGATGGTGTTATCACATTAGAAGAATCGAAAGGCTTCGCAACGG | |
| | | | AATTAGATGTTGTCGAAGGTATGCAATTTGACCGTGGTTATGTATCTCCATACATGATCACAGATACAGA | |
| | | | AAAAATGGAAGCAGTGCTTGAAAATCCATACATCTTAATTACAGATAAAAAAGTTTCTAGCATCCAAGAA | |
| | | | ATCTTGCCTATCTTAGAACAAGTAGTTCAACAAGACCGCTATTAATTATCGCAGAAGATGTCGAAGGCGA | |
| | | | AGCGCTCGCAACATTAGTCGTCAACAAACTTCGTGGTACATTCAATGCGGTAGCGGTA | |
| | | | (SEQ ID NO:95) | |
| B. megaterium | cpn60 | | GCAACAGTTTTAGCGCAAGCAATGATCAGAGAAGGTCTTAAAAACGTAACGGCTGGTGCTAACCCAATGG | |
| 899 | | | GTATCCGTAAAGGTATGGAAAAGGCAGTAGCTGTAGCGGTTGAAGAACTAAAAGCAATCTCTAAACCAAT | |
| | | | TCAAGGTAAAGATTCAATTGCTCAAGTAGCGGCTATCTCAGCAGCTGACGAAGAAGTAGGTCAATTAATT | |
| | | | GCTGAAGCAATGGAGCGCGTTGGTAACGACGGCGTTATCACACTTGAAGAATCAAAAGGTTTCACAACTG | |
| | | | AATTAGAAGTGGTAGAAGGTATGCAGTTTGACCGTGGATATGCATCTCCTTACATGGTAACTGATTCAGA | |
| | | | TAAAATGGAAGCTGTATTAGATGATCCATACATCTTAATCACAGACAAAAAAATCGGTAACATTCAAGAA | |
| | | | ATCTTACCGGTATTAGAGCAAGTTGTTCAACAAGGCAAGCCTCTATTGATCATCGCTGAAGACGTAGAAG | |
| | | | GCGAAGCTTTAGCAACATTAGTTGTGAACAAACTTCGTGGTACATTCACAGCTGTAGCTGTT | |
| | | | (SEQ ID NO:96) | |
| B. megaterium | cpn60 | ATC19213 | GCAACTGTATTAGCGCAAGCTATGATTCGTGAAGGTCTTAAAAACGTAACAGCTGGTGCTAACCCAATGG | |
| bgsc | | | GTCTTCGTAAAGGTATCGAAAAAGCTGTTACTGCTGCAATTGAAGAATTAAAAACGATTTCTAAACCAAT | |
| | | | CGAAGGCAAATCTTCTATCGCACAAGTAGCTGCTATTTCTGCAGCTGACGAAGAAGTAGGTCAATTAATC | |
| | | | GCTGAAGCAATGGAGCGCGTTGGTAACGACGGCGTTATTACTTTAGAAGAGTCTAAAGGATTCACAACAG | |
| | | | AATTAGACGTAGTAGAAGGTATGCAATTTGATCGTGGATATGCATCTCCTTACATGATTACTGATTCTGA | |
| | | | CAAAATGGAAGCAGTTCTTGATAACCCATATATCTTAATTACTGACAAAAAGATTTCTAACATTCAAGAA | |
| | | | ATCTTACCAGTATTAGAGCAAGTGGTACAACAAGGTAAACCACTTCTTATCATTGCTGAAGATGTAGAAG | |
| | | | GGCGAAGCTTTAGCTACCATTAGTAGTGAACAAACTTCGTGGTCATTCAATGTAGTAGCTGTT | |
| | | | (SEQ ID NO:97) | |
| B. coagulans | cpn60 | | GCGACCGTTCTGGCCCAGGCAATGATCCGTGAAGGCCTGAAAAACGTAACAGCAGGCGCAAACCCGGTTG | |
| CECT12 | | | GCATCCGCAAAGGGATTGAAAAAGCGGTTGCGGCTGCTGTTGAAGAATTAAAAGCCATTTCGAAACCAAT | |
| | | | CGAAGGCAAAGCTTCCATCGCCCAAGTTGCTGCAATTTCCTCTGCTGACGAAGAAGTTGGCGAATTGATC | |
| | | | GCTGAAGCAATGGAACGCGTGGGCAACGACGGCGTCATTACCATTGAAGAATCAAAAGGCTTCTCAACGG | |
| | | | AATTGGACGTTGTGGAAGGGATGCAGTTTGACCGTGGCTATGCATCGCCTTACATGGTAACGGATTCCGA | |
| | | | CAAAATGGAAGCTGTTCTGGATAACCCTTATATCTTAATTACAGACAAGAAGATTTCCAATATCCAGGAA | |
| | | | ATCCTCCCTGTTCTCGAACAAGTTGTCCAACAAGGCAAACCGCTGTTGCTGATTGCGGAAGATGTTGAAG | |
| | | | GGGAAGCTCTTGCAACACTCGTTGTCAACAAACTGCGTGGCACATTCAATGCAGTTGCGGTG | |
| | | | (SEQ ID NO:98) | |
| A. acidocal- | cpn60 | | GCGACGGTGCTGGCGCAGGCGATGATCCGCGAGGGTCTGAAGAACGTCGCCGCTGGTGCGAACCCGATGG | |
| darius | | | TGCTCCGCCGCGGCATTGAGAAGGCCGTGACGGCTGCGGTCGAGGAGCTGAAGAAGATCGCGAAGCCGGT | |
| CECT4328 | | | CCAGGGCCGCAAGAACATCGCGGAGGTTGCCGCCATCTCGGCTGGTTCGAACGAAATCGGCGAGCTCATC | |
| | | | GCGGATGCGATGGAGAAGGTTGGCAACGACGGCGTGATCACCGTCGAAGAGTCGAAGGGCTTCACGACCG | |
| | | | AGCTTGAGGTCGTCGAGGGTATGCAGTTCGACCGCGGCTACATCTCGCCGTACATGGTGACGGACGCGGA | |
| | | | CAAGATGGAGGCTGTGCTGGACGAGCCGCTCATCCTCATCACCGACAAGAAGGTCTCGAGCATCCAGGAG | |
| | | | ATCCTGCCGGTGCTGGAGCGCGTCGTGCAGGCTGGCCGTTCGCTGCTCCTCATCGCCGAGGATGTGGAGG | |
| | | | GCGAAGCGCTCGCGACGCTCGTGGTCAACAAGATCCGCGGTACGTTCAACGCCGTGGCCGTCAAA | |
| | | | (SEQ ID NG:99) | |
| B. lentus | cpn60 | | GCAACTGTTCTTGCACAAGCAATGATCCGTGAAGGCTTGAAAAACGTAACTGCTGGAGCTAATCCTGTTG | |
| CECT 18 | | | GCGTTAAAAAAGGGATGGAAAAAGCAGTTGCAACAGCAGTAACTGAGCTACAAACTATCTCAAAACAAAT | |
| | | | TGAAGATAAAGAATCAATTGCTCAAGTTGCATCTATTTCTTCTGGTGACGAAGAAGTTGGCCAATTAATA | |
| | | | GCTGAAGCAATGGAACGTGTTGGTAATGATGGCGTTATTACAATTGAAGAGTCTCGTGGTTTCACTACAG | |
| | | | AGCTTGAAGTTGTAGAAGGAATGCAGTTCGACCGTGGTTATGCATCTCCTTATATGGTAACAGATTCTGA | |
| | | | TAAAATGGAAGCTGTGCTTGAAAATCCATATATCTTGATCACAGATAAGAAAATTACAAACATCCAAGAA | |
| | | | GTACTACCTGTTCTTGAGCAAGTTGTTCAACAAGGTAAACCATTGTTGATGATTGCTGAAGATGTAGAAG | |
| | | | GTGAAGCACTTGCTACACTTGTAGTAAACAAACTTCGCGGAACATTCAACGCAGTAGCTGTT | |
| | | | (SEQ ID NO:100) | |
| H. halophilus | cpn60 | | GCAACCGTACTAGCGCAAGCGATGATCCGTGAAGGTCTAAAAAACGTAACATCCGGTGCGAACCCAGTAG | |
| | | | GCATTCGCCGCGGAATTGAAAAAGCAACCGAAGTCGCTACTCAGGAACTTCGCAAAATCTCTAAGCCAAT | |
| | | | CGAAGGCCGCGAGTCCATTTCTCAGGTAGCTTCCATCTCTGCTTCCGATAACGAAGTCGGCCAGCTGATT | |
| | | | GCTGAAGCGATGGAGCGCGTAGGAAACGATGGCGTTATTACAATTGAAGAATCTAAAGGTTTCAATACAG | |
| | | | AACTAGAAGTGGTTGAAGGTATGCAGTTCGACCGCGGCTATGCTTCTCCATACATGGTTACAGACCAGGA | |
| | | | TAAAATGGAAGCGGTTCTTGATGATCCTTACATTCTAATTACGGATAAGAAAATCAACAACATCCAGGAA | |
| | | | GTACTTCCTGTACTTGAGCAAGTGGTACAGCAATCCAAGCCGTTGCTACTGATCTCTGAAGACGTAGAAG | |
| | | | GCGAAGCACTTGCTACACTTGTTGTGAACAAACTGCGCGGTACATTCAACGCTGTATCCGTT | |
| | | | (SEQ ID NO:101) | |
| B. marinus | cpn60 | | GCAACTGTTCTTGCTCAAGCAATGATCCGTGAAGGTCTTAAAAACGTTACAGCTGGTGCAAACCCAGTTG | |
| | | | GCGTTCGTAAAGGAATTGAAAAAGCGGTTCAATCAGCACTTGTTGAGCTTAAAGAGATCTCAAAACCGAT | |
| | | | TGAAGGCAAAGAGTCGATTGCACAAGTTGCAGCTATCTCTTCATCAGATGAAGAAGTAGGGCAATTGATT | |
| | | | GCTGAAGCAATGGAGCGCGTTGGTAACGATGGCGTGATTACAATCGAAGAATCAAAAGGCTTCACAACTG | |
| | | | ACTGGATGTAGTAGAAGGTATGCAATTTGACCGTGGATATGCATCACCGTACATGGTAACAGATTCAGAT | |
| | | | AAAATGGAAGCAGTTTTAGAAAATCCATATATCTTAATCACAGACAAGAAAATCGGTAACATCCAAGAAG | |
| | | | TGCTTCCTGTACTTGAGCAAGTTGTACAACAAGGTAAGCCACTATTGATTGTTGCTGAAGATGTTGAAGG | |
| | | | CGAAGCACTAGCAACACTTGTTGTGAACAAACTACGTGGAACATTCAACGCAGTAGCTGTC | |
| | | | (SEQ ID NO:102) | |
| S. psychrophila | cpn60 | | GCAACAGTTCTAGCGCAAGCAATGATCCGTGAAGGACTGAAAAACGTAACTGCAGGTGCTAACCCTGTC | |
| CECT4073 | | | GGAATCCGTAAAGGAATCGAAAAAGCGGTTATAGCTGCTGTTGAAGGCCTTCAAGAATCTCCAATGAAA | |
| | | | TCGAAGGAAAAGAAGAGATTGCACAAGTCGCATCTATTTCTTCTGGAGACGAAGAAGTTGGGAAACTTA | |
| | | | TTGCTGAAGCAATGGAGCGCGTTGGCAACGATGGTGTCATTACTATCGAAGAGTCAAAAGGCTTCACGA | |
| | | | CTGAACTAGACGTTGTTGAAGGAATGCAATTTGACCGCGGTTATGCATCTGCATACATGGCAACGGATA | |
| | | | CAGACAAAATGGAAGCAGTTTTGGACAATCCGTATATCTTGATCACAGATAAAAAGATTACGAACATCC | |
| | | | AAGAAATTCTTCCTGTTCTTGAGCAAGTAGTTCAACAAGGTAAGCCACTTCTTATGATCGCAGAAGACG | |
| | | | TTGAAGGCGAAGCACTTGCAACACTTGTTGTGAACAAACTACGTGGTACATTCAATGCTGTTGCTGTT | |
| | | | (SEQ ID NO:103) | |
| P. macerans | cpn60 | | GCAACAGTTCTTGCTCAGGCAATGATCCGTGAAGGCCTTAAGAACGTAACTGCAGGTGCTAACCCAATGG | |
| CECT19 | | | GCATCCGCAAAGGAATTGAAAAAGCGGTTTCTACTGCTGTTGAAGAGTTAAAAGCTATTTCAAAACCTAT | |
| (= B58 | | | CGAAAACAAAGAATCTATCGCACAGGTTGCTGCTATTTCTGCTGCTGACAATGAAGTTGGCCAGCTGATC | |
| B. firmus | | | GCTGAAGCAATGGAGCGCGTTGGCAACGATGGTGTTATCACAATCGAAGAATCTAAAGGTTTCACAACTG | |
| | | | AGCTTGATGTGGTAGAAGGTATGCAATTCGACCGCGGATACGCTTCACCATACATGGTTACAGATTCTGA | |
| | | | TAAGATGGAAGCGGTTCTTGAAAACCCTTATATCTTAATCACTGATAAGAAGATCACAAGCATCCAGGAA | |
| | | | ATTCTTCCTGTACTTGAGCAGGTTGTACAGCAAGGCAAGCCTTTATTGCTTGTAGCTGAGGATGTTGAAG | |
| | | | GTGAAGCACTAGCTACATTAGTAGTGAATAAGCTTCGTGGAACTTTCAACGCTGTAGCGGTT | |
| | | | (SEQ ID NO:104) | |
| B. psychro- | cpn60 | | GCTACTGTCCTTGCACAAGCTATGATTCGTGAAGGCCTGAAAAACGTAACGGCTGGCGCGAATCCTATGG | |
| saccharolyticus | | | GCATTCGTAAAGGGATTGAAAAAGCTGTGAAAGCTGCAATTAGTGAGTTACAAGCTATCTCTAAACCAAT | |
| CECT | | | CGAAAACAAAGAGTCTATTGCACAAGTTGCAGCAATCTCAGCTTCTGACGAAGAAGTGGGTCAATTAATT | |
| 4074 | | | GCTGAAGCAATGGAACGCGTTGGCAACGACGGTGTTATCACAATTGAAGAGTCTAAAGGATTCTCAACTG | |
| | | | AATTGGACGTAGTAGAAGGTATGCAGTTCGACCGTGGATATGCATCTGCTTATATGGTAACAAACCCAGA | |
| | | | TAAAATGGAAGCAGTTCTTGAAAATCCATATATCTTAATTACTGACAAAAAAATCTCAAACATTCAAGAA | |
| | | | ATTCTTCCTGTACTTGAACAAGTTGTTCAACAAGGAAAATCTCTATTGCTAATTGCTGAAGACATTGAAG | |
| | | | GCGAAGCACTATCAACACTTGTTGTGAACAAACTTCGTGGAACATTCAATGCAGTTGCTGTA | |
| | | | (SEQ ID NO:105) | |
| B. mycoides | cpn60 | | GCAACTGTATTAGCGCAAGCTATGATTCGTGAAGGTCTTAAAAACGTAACAGCTGGTGCAAACCCAATGG | |
| CECT 4128 | | | GTCTTCGTAAAGGTATCGAAAAAGCTGTTACTGCTGCAATTGAAGAATTAAAAGCGATTTCTAAACCAAT | |
| | | | CGAAGGTAAATCTTCTATCGCACAAGTAGCTGCTATTTCTTCGGCTGACGAGAAGTAGGTCAATTAATC | |
| | | | GCTGAAGCAATGGAGCGCGTTGGTAACGACGGCGTTATTACTTTAGAAGAATCTAAAGGATTCACAACAG | |
| | | | AATTAGACGTAGTAGAAGGTATGCAATTTGATCGTGGATAAGCATCTCCTACATGATTACTGATTCTGAC | |
| | | | AATGAGAGTTCACTTCTTATCATTGCTGAAGATGTAGAAGGCGAAGCGTTAGCTACATTAGTAGTGAACA | |
| | | | AACTTCGTGGTACATTCAATGTAGTTGCTGTT (SEQ ID NO:106) | |
| B. alcalophilus | cpn60 | | GCGACTGTTCTAGCTCAAGCGATGATTCGTGAAGGTCTTAAAAACGTAACATCTGGTGCGAACCCAATGG | |
| CECT 1 | | | GTATCCGTAAAGGGATTGAAAAAGCAACAGCTGCTGCGGTTACAGAACTTAAAAATATTGCGAAACCAAT | |
| | | | CGAAGGCAAAGAGTCAATCGCACAAGTTGCGGCTAACTCAGCAGCTGACGAAGAAGTTGGACAAATTATC | |
| | | | GCAGAAGCAATGGAACGTGTTGGAAACGACGGCGTTATTACAATCGAAGAATCAAAAGGTTTCTCTACTG | |
| | | | AATTAGAAGTAGTAGAAGGTATGCAATTCGATCGTGGTTTCGTTTCTCCATACATGGTAACCGATTCTGA | |
| | | | CAAAATGGAAGCAGTTCTTGAAAATCCATATATTTTAATTACGGATAAAAAGATTGCAAGCATTCAAGAA | |
| | | | ATCCTACCAGTTCTTGAGCAAGTGGTTCAACAAGGTAAACCAATCCTAATCATCGCTGAAGATGTTTGAA | |
| | | | GGGGAAGCTCAAGCAACATTAGTTGTTAATAAATTACGTGGTACATTCAATGCGGTAGCCGTT | |
| | | | (SEQ ID NO:107) | |
| A. aneruino- | cpn60 | ATCC12856 | GCTACAGTTCTTGCTCAAGCGATGATTCGCGAAGGCTTGAAAAACGTAACAGCGGGTGCAAACCCGATGG | |
| lyticus | | | TTATGCGCAAAGGTATCGAAAAGGCAGTTCGTGCAGCAGTAGAAGAACTGCATGCGATTTCTAAACCAAT | |
| | | | CGAAGGTAAAGAATCTATCGCACAAGTAGCAGCTATTTCTGCTGCTGATGAGGAAATCGGCCAACTGATT | |
| | | | GCTGAAGCTATGGAAAAAGTAGGAAAAGATGGCGTTATCACAGTAGAAGAATCCAAAGGCTTCACAACAG | |
| | | | AACTTGATGTTGTAGAAGGTATGCAATTCGACCGCGGATACGCTTCTCCATACATGATCACGGATACTGA | |
| | | | TAAGATGGAAGCAGTGCTTGATAATCCGTATATCTTGATTACGGATAAGAAAATCTCTAACATTCAGGAA | |
| | | | ATCCTTCCTGTGTTAGAGAAAGTTGTACAACAAGGCAAGCCGCTTGTTATCATCGCTGAAGATGTAGAAG | |
| | | | GCGAAGCACTGGCTACGCTTGTTGTAAATAAATTGCGTGGTACATTTACTGCGGTAGCAGTA | |
| | | | (SEQ ID NO:108) | |
| A. xylanus | cpn60 | ATCC | GCAACAGTTTTAGCACAAGCAATGATTAAGAAGGATTGAAAAACGTTGCTTCTGGACCAAACCCTGTCG | |
| | | 51415 | GTGTTCGCCGTGGAATTGAAAAAGCTGTTGAAGTTGCAGTAGACGAGCTTAGAAAAATTTCACAAACAG | |
| | | | TTGAAGATAAAGAATCAATCGCTCAAGTTGCAGCTATTTCAGCAAATGACGAAGAAGTAGGTCAATTAA | |
| | | | TCGCTGAAGCAATGGAGCGCGTTGGTAAGATGGTGTAATTACTGTTGAAGAATCAAGAGGATTCAGCAC | |
| | | | TGAACTTGAAGTAGTAGAAGGTATGCAATTTGACCGCGGATATACTTCACCATATATGGTATCTGACCA | |
| | | | AGATAAGATGGAAGCAGTGCTTGAAGATCCATATATTTTAGTAACAGATAAGAAATTAACACATTCAAG | |
| | | | ATGTATTACCAGTACTTGAGCAAGTTGTACAACAAAGCAAGCCACTATTAATTATTGCTGAAGATGTTG | |
| | | | AAGGTGAAGCACTTGCAACATTGGTTGTAAACAAACTTCGTGGAACATTTAATGCAGTAGCTGTA | |
| | | | (SEQ ID NO:109) | |
| V. pantothen- | cpn60 | ATCC14576 | GCAACTGTATTAGCTCAGTCCATGATTCGTGAAGGTCTTAAACGTAGCATCCGGTGCTAACCCTGTTGG | |
| ticus (=B65 | | | TGTTCGCCGCGGAATCGAAAAGGCTGTTGAAGTAGCAGTAAAAGAACTAAAAATATTTCCAAGTCAATC | |
| B. panthothen- | | | GAAAGCAAGGAATCTATTGCTCAAGTAGCAGCAGTTTCTTCTGACGATGCAGAAGTTGGTAAGTTAATT | |
| ticus) | | | TCTGAAGCAATGGAACGTGTTGGTAACGACGGAGTTATTACTATTGAAGAATCAAAAGGTTTCAACACA | |
| | | | GAGCTAGAAGTAGTTGAAGGTATGCAATTTGACCGTGGATATGCTTCTCCATACATGGTAACAGACCAA | |
| | | | GACAAAATGGAAGCAGTTTTGGAAAATCCGTACATCCTAATTACGGATAAGAAAATTGGTAACATTCAA | |
| | | | GAAGTATTACCTATACTTGAACAAGTTGTACAGCAAGGAAAACCTTTATTGATGATTGCTGAGGATGTA | |
| | | | GAAGGCGAAGCGCTTGCTACATTAGTAGTTAACAAATTGCGTGGAACATTCAATGCAGTAGCTGTA | |
| | | | (SEQ ID NO:110) | |
| P. popillae | cpn60 | | GCTACGGTTCTGGCTCAAGCGATGATTCGCGAAGGCTTGAAGAACGTTACGGCTCGCGCGAATCCGATG | |
| | | | GTCGTTCGCATCAAGGGATCGAGAAAGCAGTGAAANCCGCTGTTGAAGATCTGAAGAAAATTGCGAAGC | |
| | | | CAATTGAAAACAAGCAAGCATCGCTCAAGTTGCTGCAATCTCTNCCGATGACGAAGAAGTCGGCACATT | |
| | | | GATCGCAGAAGCAATGGAGAGAGTCGGCAATGACGGTGTAATTACGGTTGAGGAATCCAAAGGCTTCAA | |
| | | | TACGGAGCTTGAAGTTGTAGAAGGGATGCNATTNGACCNTGGCTNTNTATCTCCGTACATGATCACGGA | |
| | | | TACGGACAAGATGGAAGCTATCCTCGATACCCCATATATCTTGATCACAGATAAGAAGGTTTCCAACAT | |
| | | | TCAAGAAATCCTTCCTGTTCTTGAGAAAGTCATTCAACAAGGCAAGCAGCTCCTGATCATCGCTGAGGA | |
| | | | TGTAGAAGGCGAGCTCAAGCAACCTTGATCTTGAATAAGCTTGCGGACATTCACTTGCGTTGCCGTTA | |
| | | | (SEQ ID NO:111) | |
| S. pyogenes | cpn60 | ATCC19615 | GCAACAGTTTTGACACAAGCCATTGTTCATGAAGGACTAAAAAATGTGACAGCAGGTGCTAATCCAATT | |
| | | | GGTATCCGTCGAGGCATTGAAACAGCAACAGCAACAGCCGTTGAAGCCTTGAAAGCCATTGCTCAACCT | |
| | | | GTATCTGGCAAGGAAGCTATTGCTCAGGTCGCTGCAGTATCATCACGCTCTGAAAAAGTTGGAGAGTAT | |
| | | | ATCTCAGAAGCTATGGAGCGTGTGGGCAACGATGGTGTGATTACCATCGAAGAATCTCGAGGTATGGAA | |
| | | | ACAGAACTTGAAGTG0TTGAAG0CATGCATTTGACCGTGGTTACCTGTCTCAATACATGGTCACAGACA | |
| | | | ATGAAAAAATGGTTGCAGACCTTGAAAACCCATTTATCTTGATCACGGATAAAAAAGTGTCAAACATCC | |
| | | | AAGACATTTTGCCACTACTTGAGGAAGTTCTTAAAACCAACCGTCCATTACTCATTATTGCAGATGATG | |
| | | | TGGATGGTGAGCCCTTCCAACCCTTGTCTTGAACAAGATTCGTGGTACTTTCAATGTGGTTGCTGTC | |
| Escherichia- | cpn60 | | GCAACCGTACTGGCTCAGGCTATCATCACTGAAGGTCTGAAAGCTGTTGCTGCGGGCATGAACCCGATG | |
| coli K12 | | | GACCTGAAACGTGGTATCGACAAAGCGGTTACCGCTGCAGTTGAAGAACTGAAAGCGCTGTCCGTACCA | |
| | | | TGCTCTGACTCTAAAGCGATTGCTCAGGTTGGTACCATCTCCGCTAACTCCGACGAAACCGTAGGTAAA | |
| | | | CTGATCGCTGAAGCGATGGACAAAGTCGGTAAAGAAGGCGTTATCACCGTTGAAGACGGTACCGGTCTG | |
| | | | CAGGACGAACTGGACGTGGTTGAAGGTATGCAGTTCGACCGTGGCTACCTGTCTCCTTACTTCATCAAC | |
| | | | AAGCCGGAAACTGGCGCAGTAGAACTGGAAAGCCCGTTCATCCTGCTGGCTGACAAGAAAATCTCCAAC | |
| | | | ATCCGCGAAATGCTGCCGGTTCTGGAAGCTGTTGCCAAAGCAGGCAAACCGCTGCTGATCATCGCTGAA | |
| | | | AGATGTAGAAGGCGAAGCGCTGGCAACTCTGGTTGTTAACACCATGCGTGGCATCGTGAAAGTCGCTGC | |
| | | | GGTT (SEQ ID NO:113) | |
| Brassica- | cpn60 | | TCTGTGGTTCTTGCACAAGGTTTTATTGCTGAGGGTGTCAAGGTGGTGCCTGCTGGTGCAAACCCTGTA | |
| napus | | | TTGATCACTAGAGGCATTGAGAAGACAGCAAAGGCTTTGGTAGCCGAGCTCAAGAAAATGTCTAAGGAG | |
| chloroplast | | | GTTGAAGACAGTGAGCTTGCAGATGTGGCAGCCGTTAGTGCCGGTAACAATGCAGAAATTGGAAGCATG | |
| beta | | | ATTGCTGAAGCAATGAGCAGAGTGGGCAGGAAGGGTGTGGTGACACTTGAGGAGGGTAAAAGTGCAGAG | |
| | | | AACGCTCTCTACGTGGTGGAAGGAATGCAATTTGATCGAGGTTATGTCTCCCCTTACTTTGTGACAGAC | |
| | | | AGCGAGAAAATGTCAGTTGAGTTCGACAATTGCAAGTTGCTTCTTGTTGACAAGAAAATTACCAATGCA | |
| | | | AGGGATCTTGTTGGTGTTCTGGAGGATGCAATTAGAGGAGGATACCCAATTTTAATAATTGCGGAAGAC | |
| | | | ATTGAGCAGGAGGCTTTAGCGACCCTTGTTGTTAACAAGCTTAGAGGCACACTGAAGATTGCAGCTCTC | |
| | | | (SEQ ID NO:114) | |

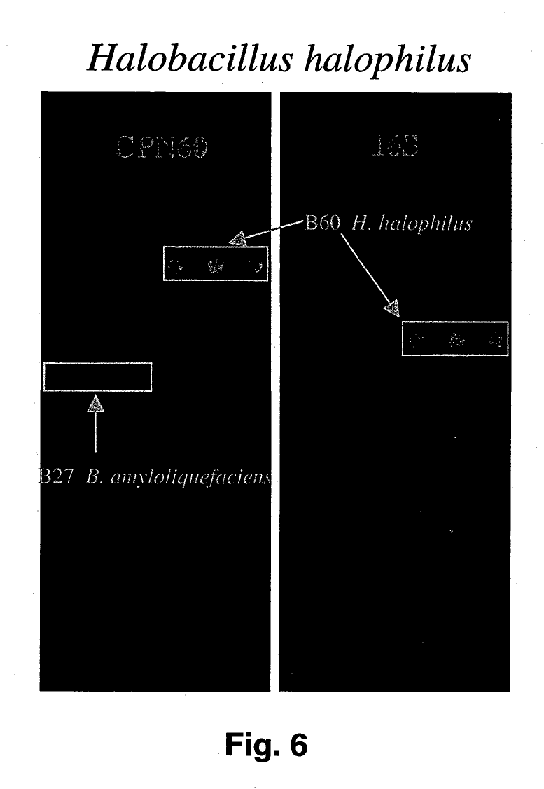

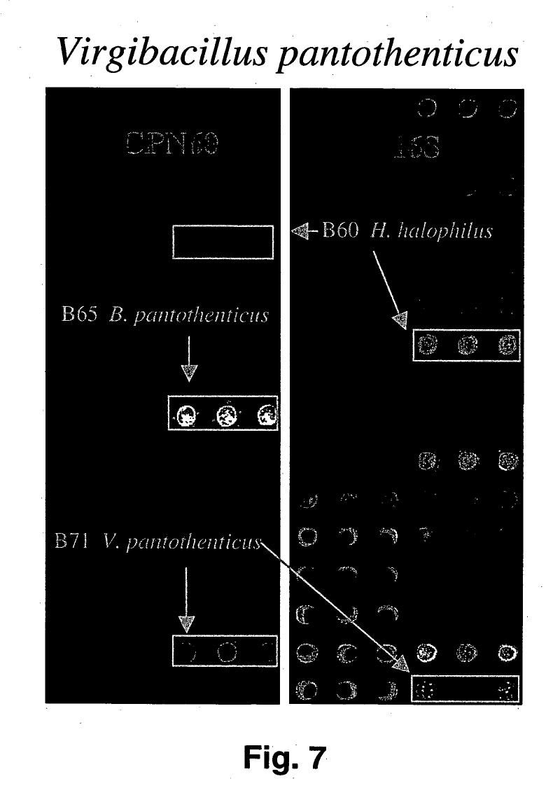

Within this simple system of 16S and cpn60 amplicons from a single species hybridized to amplicon probes of perfect match on the array, the dual backbone prototype was easily able to distinguish three of the four species tested in this assay. H. halophilus gave a strong signal only for its matching 16S and cpn60 probes (FIG. 6). A. xylanus gave a strong signal for the 16S of H. halophilus in addition to its matching 16S probe (FIG. 4). However, the only cpn60 signal came from the probe for A. xylanus. There was no strong signal for its corresponding 16S probe, when V. pantothenticus was hybridized, due to irregularities in the printed DNA spot (FIG. 7). However, two cpn60 probes gave signals—B. pantothenticus and V. pantothenticus. A closer look at these two amplicons revealed that the sequences were identical. Even with an identical sequence, the signal was significantly stronger for the B. pantothenticus than for the V. pantothenticus, which had less DNA deposited on the array (determined by a deoxynucleotidyl terminal transferase assay).

The above three hybridizations were all done with Bacillus-like species that have been reclassified into new genera based on a significant difference from the core Bacillus species. B. amyloliquefaciens (FIG. 5) gave several signals for the 16S probes. It appeared that the B. amyloliquefaciens probe was the strongest, but it was difficult to confirm due to spot irregularities (the mooning effect). When examining the cpn60 probes, signals were obtained from B. subtilis and B. amyloliquefaciens. A closer look at the cpn60 for B. subtilis showed a 6% difference in sequence similarity, which is believed to be too close to discriminate using microarrays. In this case the dual backbone array was able to identify the sample as a Bacillus, and even narrow it down to a pair of species, but it was not able to positively identify it as B. amyloliquefaciens.

From the results obtained above, it was concluded that the optimal hybridization temperature varied between the 16S and cpn60 amplicons, but a compromise at 55° C. at which both types of amplicons hybridized with adequate specificity was appropriate.

By simultaneously assaying for virulence and antimicrobial resistance genes on the same microarray a significant reduction of effort and time were achieved.

The oligonucleotide microarray of the present invention is a powerful tool for the detection of virulence and antimicrobial resistance genes in E. Coli strains.

In accordance with the present invention, it is the first time according to the inventors that two different types of taxonomic sequences (16S and cpn60) have been used together and the results analyzed jointly to obtain corroboration that in some case it is not possible to have otherwise. It is also the first time that antibiotic resistance genes have been used with virulence genes in E. coli on the same array to obtain, in one experiment, information on the nature of the pathogen and how best to treat it. It is also the first time that many variants of the genes probes for virulence are being disclosed to pinpoint the precise type and, in some cases, the target species of the pathogen detected. Thus through a combination of probes, the inventors achieve a better and faster results than previously possible with DNA microarrays of the prior art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. An array comprising:
a) a substrate; and
b) a plurality of nucleic acid probes specifically and simultaneously recognizing the presence of a plurality of different genes, each of said probes being bound to said substrate at a discrete location; said plurality of probes comprising a first probe for detecting a first gene of a species of a microorganism and at least another probe for detecting at least one other gene of said species or of a different species of a microorganism.

2. The array of claim 1, comprising at least two different probes specific for a single gene.

3. The array of claim 2, wherein said array comprises a subarray containing said at least two probes at adjacent discrete locations on said substrate.

4. The array of claim 1, wherein said first probe is specific for a virulence gene or a fragment thereof or a sequence substantially identical thereto, and said at least one other probe is specific for an antibiotic resistance gene.

5. The array of claim 1, wherein said first probe is specific for a variant of a virulence gene or a fragment thereof or a sequence substantially identical thereto, and said at least one other probe is specific for an antibiotic resistance gene, said first probe allowing detection of different types and/or species of microorganism.

6. The array of claim 1, wherein said microorganism is a bacterium.

7. The array of claim 6, wherein said bacterium is of the family Enterobacteriaceae.

8. The array of claim 7, wherein said bacterium is E. coli.

9. The array of claim 4, wherein said virulence gene encodes a polypeptide of a class of proteins selected from the group consisting of toxins, adhesion factors, secretory system proteins, capsule antigens, somatic antigens, flagellar antigens, invasins, autotransporter proteins, and aerobactin system proteins.

10. The array of claim 1, wherein said different genes are selected from the group consisting of Tem, Shv, oxa-1, oxa-7, pse-4, ctx-m, aht(3″)-Ia (aadA1), ant(2″)-Ia (aadB)^{b}, aac(3)-IIa (aacC2), aac(3)-IV, aph(3′)-Ia (aphA1), aph(3′)-IIa (aphA2), tet(A), tet(B), tet(C), tet(D), tet(E), tet(Y), catI, catII, catIII, floR, dhfrI, dhfrV, dhfrVII, dhfrIX, dhfrXIII, dhfrXV, suII, suII, intégron classe 1 3′-CS, vat, vatC, vatD, vatE, vga, vgb, and vgbB,

11. The array of claim 1, wherein said plurality of nucleic acid probes are sequences selected from the group consisting of SEQ ID NO:1 to SEQ ID NO:64, or a fragment thereof, or a sequence having at least 50% identity, preferably at least 70% identity, more preferably having 80% identity and most preferably having 90% identity with said sequences.

12. The array of claim 1, wherein said plurality of different genes are selected from the group consisting of 16S genes, genes encoding heat shock proteins, genes encoding RNA polymerase, genes encoding DNA gyrases, genes encoding lipases, genes encoding cellulases, genes encoding proteases, genes of clinical interest, genes encoding virulence factors, genes encoding growth factors, and genes encoding toxins.

13. The array of claim 1, wherein said first probe is specific for a 16S gene or a fragment thereof or a sequence substantially identical thereto, and said at least one other probe is specific for cpn60 gene.

14. A method of detecting the presence of a microorganism in a sample, said method comprising the steps of:
a) contacting the array of claim 1 with a sample nucleic acid of said sample; and
b) detecting association of said sample nucleic acid to a probe on said array;
wherein association of said sample nucleic acid with said probe is indicative that said sample comprises a microorganism from which the nucleic acid sequence of said probe is derived.

15. The method of claim 14, wherein said method further comprises extracting said sample nucleic acid from said sample prior to contacting said sample nucleic acid with said array.

16. The method of claim 14, wherein said sample is selected from the group consisting of environmental sample, biological sample and food.

17. The method of claim 16 wherein said environmental sample is selected from the group consisting of water, air and soil.

18. The method of claim 16, wherein said biological sample is selected from the group consisting of blood, urine, amniotic fluid, feces, tissues, cells, cell cultures and biological secretions, excretions and discharge.

19. The method of claim 14, wherein said sample is a tissue, body fluid, secretion or excretion from a subject.

20. A method for determining a pathotype of a species of a microorganism in a sample, said method comprising the steps, of:
a) contacting the array of claim 1 with a sample nucleic acid of said sample; and
b) detecting association of said sample nucleic acid to a probe on said array;
wherein association of said sample nucleic acid with said probe is indicative that said sample having a pathotype from which the nucleic acid sequence of said probe is derived.

21. The method of claim 21, further comprising the step of:
c) tabulating results for most abundant species based on intensity of the association detected.

22. A method for diagnosing an infection by a microorganism in a subject, said method comprising the steps of:
a) contacting the array of claim 1 with a sample nucleic acid of said sample; and
b) detecting association of said sample nucleic acid to a probe on said array;
wherein association of said sample nucleic acid with said probe is indicative that said sample has been infected by a microorganism from which the nucleic acid sequence of said probe is derived.

23. A kit comprising the array of claim 1 together with instructions for use thereof.

24. The kit of claim 23, wherein said use is for at least one of:
(a) detecting the presence of a microorganism in a sample;
(b) determining the pathotype of a microorganism in a sample;
(c) diagnosing an infection by a microorganism in a subject;
(d) diagnosing a condition related to infection by a microorganism, in a subject;
(e) characterizing a microbial complex sample or microbial community on a one-time basis; and
(f) following the evolution over time of a microbial complex sample or microbial community. This may include comparison between different batches of commercial products based on complex microbial samples, comparison between similar products from different suppliers and monitoring the bacterial composition of commercial products over storage time.
